(19) United States
(12) Patent Application Publication
(10) Pub. No.: US 2020/0197349 A1
(43) Pub. Date: Jun. 25, 2020

(54) COGNITIVE FUNCTION

(71) Applicant: Howard Foundation Holdings Ltd, Great Shelford (GB)

(72) Inventors: Alan Norman HOWARD, Great Shelford (GB); Rebecca POWER, West Campus, Waterford (IE); John NOLAN, West Campus, Waterford (IE)

(73) Assignee: Howard Foundation Holdings Ltd, Great Shelford (GB)

(21) Appl. No.: 16/795,058

(22) Filed: Feb. 19, 2020

Related U.S. Application Data
(63) Continuation-in-part of application No. 16/426,771, filed on May 30, 2019.
Application No. 16/426,771 is a continuation-in-part of application No. 15/980,083, filed on May 15, 2018.

(51) Int. Cl. A61K 31/23; A61K 31/045; A61K 9/00; A61P 25/00

(52) U.S. Cl. CPC A61K 31/23; A61P 25/00; A61K 9/0053; A61K 31/045

(57) ABSTRACT

Disclosed is a method of improving the cognitive function of a cognitively healthy human adult, the method comprising administering to the human adult an effective amount of a composition comprising lutein, zeaxanthin and meso-zeaxanthin and at least one omega-3 fatty acid, over a period of at least 24 months, the improved cognitive function being a detectable improvement in: spatial working memory as measured by the CANTAB Connect Research software SWM task; and/or reduced reaction time as measured by the CANTAB Connect Research Software RTI task.

### CROSS-REFERENCE TO RELATED APPLICATIONS

This application is a continuation-in-part of U.S. application Ser. No. 16/426,771, filed May 30, 2019, which is a continuation-in-part of U.S. application Ser. No. 15/980,083, filed May 15, 2018 in the U.S. Patent and Trademark Office. This application claims the benefit of United Kingdom Application No. 1915485.5, filed Oct. 25, 2019, and United Kingdom Application No. 1720119.5, filed Dec. 4, 2017 in the United Kingdom Intellectual Property Office. All disclosures of the documents named above are incorporated herein by reference.

### FIELD OF THE INVENTION

The present invention relates to compositions for use in improving the cognitive function of adult human subjects, and more especially for use in elderly subjects (65 years or older), and in particular in elderly subjects who are cognitively healthy for their age. The invention also relates to methods of improving or maintaining the cognitive function of adult human subjects.

### BACKGROUND OF THE INVENTION

Concomitant with the growing population and increases in life expectancy in most regions is the increasing prevalence of age-related diseases. Dementia represents the most significant stage of cognitive decline and is one of the fastest growing age-related diseases. Estimates suggest that there are over 52 million adults worldwide with dementia, with prevalence numbers expected to double every 20 years (Alzheimer's Disease International, 2018). Dementia poses one of the greatest health care challenges of our society in terms of personal, societal and financial implications. Current pharmacological interventions for Alzheimer's disease (AD), the most common form of dementia, are aimed at neurochemical imbalance and have limited success in stabilising or slowing the progression of the disease. Thus, emphasis is being placed on strategies to promote healthy ageing, with the aim of minimising the burden of disability and disease in later life and maximising the quality of life for individuals in their later years.

Alzheimer's disease is the most common type of dementia, followed by vascular dementia. In the UK, the number of people with AD at present is around 860,000 and in the USA is about 5.6 million. With increasing age and population, it is anticipated that the overall prevalence of the disease will continue to increase. Despite considerable research effort, the cause of the disease is still unknown but established risk factors include age, family history of disease and education [Alzheimer's Association (2017) 2017 Alzheimer's disease facts and figures, Alzheimer's Association, Chicago, Ill., USA] and putative risk factors include cigarette smoking, physical inactivity and social isolation [Livingstone et al., (2017) Dementia prevention, intervention, and care. Lancet 390, 2673-2734]. The effectiveness of current pharmacological treatments (medications) is strictly limited and varies among individuals. Moreover, the effect of current medications is at best only palliative, as they cannot halt disease progression. The UK NHS suggests, as preventative, the following: cessation of smoking, limiting alcohol consumption, a healthy well balanced diet, and staying physically and mentally active.

There have been numerous studies on the effects of the Mediterranean Diet, a diet characterised by a high intake of vegetables, olive oil, and a moderate intake of fish, dairy products and wine. There is a consensus that adherence to such diets is associated with better cognitive performance [see e.g.: Tangney et al., (2011) Adherence to a Mediterranean-type dietary pattern and cognitive decline in a community population. Am J Clin Nutr 93, 601-607; Feart et al., (2009) Adherence to a Mediterranean diet, cognitive decline, and risk of dementia. J.A.M.A. 302, 638-648; Trichopoulou et al., (2015) Mediterranean diet and cognitive decline over time in an elderly Mediterranean population. Eur. J Nutr. 54, 1311-1321; Pelletier et al., (2015) Mediterranean diet and preserved brain structural connectivity in older subjects. Alzheimers Dement 11, 1023-1031; McEvoy et al., (2017) Neuroprotective Diets Are Associated with Better Cognitive Function: The Health and Retirement Study. J IAm Geriatr Soc 65, 1857-1862; and Lourida et al., (2013) Mediterranean diet, cognitive function, and dementia: a systematic review. Epidemiology 24, 479-489] and a reduced risk of dementia, especially AD [see e.g.: Psaltopoulou et al., (2013) Mediterranean diet, stroke, cognitive impairment, and depression: A meta-analysis. Ann. Neurol 74, 580-591; Singh et al., (2014) Association of Mediterranean diet with mild cognitive impairment and Alzheimer's disease: a systematic review and meta-analysis. J Alzheimers Dis 39, 271-282; Scarmeas et al., (2006) Mediterranean diet and risk for Alzheimer's disease. Ann Neurol 59, 912-921; Scarmeas et al., (2009) Mediterranean diet and mild cognitive impairment. Arch Neurol 66, 216-225; and Sofi et al., (2014) Mediterranean diet and health status: an updated meta-analysis and a proposal for a literature-based adherence score. Public Health Nutr 17, 2769-2782], but no clear evidence of the exact foodstuffs responsible.

Some studies have pointed to an effect from omega-3 fatty acids, of which docosahexaenoic acid (DHA) is found in high concentration in fish oil and in fatty fish such salmon and herring. There is a substantial concentration of DHA in the human brain where it forms a structural component within this neural tissue. High consumption of omega-3 fatty acids is associated with better cognitive performance and a reduced risk of dementia (see Muldoon et al., (2014) Long-chain omega-3 fatty acids and optimization of cognitive performance. Mil Med 179, 95-105; Cole et al., (2005) Prevention of Alzheimer's disease: Omega-3 fatty acid and phenolic anti-oxidant interventions. Neurobiol Aging 26 Suppl 1, 133-136; Fotuhi et al., (2009) Fish consumption, long-chain omega-3 fatty acids and risk of cognitive decline or Alzheimer disease: a complex association. Nat Clin Pract Neurol 5, 140-152; Thomas et al., (2015) Omega-3 Fatty Acids in Early Prevention of Inflammatory Neurodegenerative Disease: A Focus on Alzheimer's Disease. Biomed Res Int 2015, 172801; and Cole & Frautschy et al., (2010) DHA may prevent age-related dementia. J Nutr 140, 869-874]. However, interventional studies have shown conflicting results, with some demonstrating improvements in cognition [see Abubakari et al., (2014) Omega-3 fatty acid supplementation and cognitive function: are smaller dosages more beneficial? Int J Gen Med 7, 463-473 and Yurko-Mauro et al., (2010) Beneficial effects of docosahexaenoic acid on cognition in age-related cognitive decline. Alzheimers Dement 6, 456-464], and others demonstrating no beneficial effect [see Phillips et al., (2015) No Effect of Omega-3 Fatty Acid Supplementation on Cognition and Mood in Individuals with Cognitive Impairment and Probable Alzheimer's Disease: A Randomised Controlled Trial. Int J Mol Sci 16, 24600-24613, and van de Rest et al., (2008) Effect of fish oil on cognitive performance in older subjects: a randomized, controlled trial. Neurology 71, 430-438].

Other food items of interest include the carotenoids lutein (L) and zeaxanthin (Z). These carotenoids, which are of dietary origin are found in certain fruits and vegetables (e.g. spinach, broccoli, peppers, melon). In humans, L and Z are found in high concentrations in the eye (specifically the centre part of the retina known as the macula, where they are referred to as “macular pigment” or “macular carotenoids”) and brain [Johnson et al., (2013) Relationship between Serum and Brain Carotenoids, alpha-Tocopherol, and Retinol Concentrations and Cognitive Performance in the Oldest Old from the Georgia Centenarian Study. J Aging Res 2013, 951786; and Craft et al., (2004) Carotenoid, tocopherol, and retinol concentrations in elderly human brain. J Nutr Health Aging 8, 156-162]. High carotenoid intake has been found to result in a reduced risk of AD [Feart et al., (2016) Plasma Carotenoids Are Inversely Associated With Dementia Risk in an Elderly French Cohort. J Gerontol A Biol Sci Med Sci 71, 683-688; Min & Min (2014) Serum lycopene, lutein and zeaxanthin, and the risk of Alzheimer's disease mortality in older adults. Dement Geriatr Cogn Disord 37, 246-256; and Loef & Walach (2012) Fruit, vegetables and prevention of cognitive decline or dementia: a systematic review of cohort studies. J Nutr Health Aging 16, 626-630]. Some studies administering L and Z have shown improvement in different domains of cognition [Hammond et al., (2017) Effects of Lutein/Zeaxanthin Supplementation on the Cognitive Function of Community Dwelling Older Adults: A Randomized, Double-Masked, Placebo-Controlled Trial. Front Aging Neurosci 9, 254; and Johnson et al., (2008) Cognitive findings of an exploratory trial of docosahexaenoic acid and lutein supplementation in older women. Nutr Neurosci 11, 75-83], while another randomised trial showed no benefit [Chew et al., Age-Related Eye Disease Study 2 Research G (2015) Effect of Omega-3 Fatty Acids, Lutein/Zeaxanthin, or Other Nutrient Supplementation].

Another macular carotenoid is meso-zeaxanthin (MZ), which is a stereoisomer of zeaxanthin. The chemical structures of L, Z and MZ are shown in FIG. 1.

Vitamin E is also present in the brain, and high plasma concentrations of vitamin E have been associated with a reduced risk of AD (Mangialasche et al., (2010) High plasma levels of vitamin E forms and reduced Alzheimer's disease risk in advanced age. J Alzheimers Dis 20, 1029-1037). There have been no reports of successful treatment of AD following administration of this vitamin.

In summary, there is general agreement that there are substances in the brain which might possibly play a role in preventing in cognitively healthy subjects AD, but attempts so far to identify or use them have been unsuccessful.

Compositions comprising all three macular carotenoids (L, Z and MZ) are commercially available as nutritional supplements. One example of such a supplement is sold under the trade mark MacuPrime®, and consists of capsules containing the three macular s carotenoids L, Z and MZ in the amounts of 10 mg, 2 mg and 10 mg, respectively, per capsule. WO 2013/005037 discloses the use of such a composition for improving the visual performance of a human subject, the composition optionally further comprising a fish oil and/or an omega 3 fatty acid.

US2016/0067203 (Lion Corporation) discloses and claims compositions for improving cognitive function, the compositions comprising docosahexaenoic acid (DHA) in combination with lutein, zeaxanthin and capsanthin. The specification includes the results of a “Passive Avoidance Test” performed using mice fed the test composition or a control composition for 3 months (see especially Example 5 in Table 2 of the prior art document). The mice were given a mild electric shock when they went into a darkened compartment, and learned to avoid the darkened compartment when the test was repeated. The prior art specification also presents data (Table 3) suggesting that the amount of amyloid P protein in the brain was reduced in the test mice compared to the controls (although the numbers of animals involved in this experiment were very small, n=3 and n=4), so these results are not statistically significant.

There are no data from experiments with human subjects. Also, there is no evidence that the exemplified composition could improve cognitive function in, or stabilise the condition of, subjects already suffering from cognitive impairment.

WO2006/16755 (Trustees of Tufts College) discloses and claims a composition having “synergistic amounts of lutein and DHA for use in improvement of cognitive function”. The claims are based on a trial in which 50 human subjects (all female) were given daily supplements, over a 4 month period, containing (i) a placebo; or (ii) DHA or lutein; or (iii) DHA and lutein in combination. Various cognitive function tests were performed at start and end of the period of supplementation, and the results are shown in Table 2 of the prior art document. All compositions, except the placebo, gave statistically significant (<0.05) improvements in a verbal fluency test (hence, there was no evidence of any synergy between the DHA and lutein).

In addition however, subjects given the DHA/lutein combination demonstrated statistically significant improvements in a Shopping List Memory Test and an MIR (“Memory-in-Reality”) Apartment Test. However for the latter, it is clear from FIG. 4 of the prior art publication that the baseline score was unusually low for that group, so a big improvement was perhaps nothing to do with the supplementation. Finally, there is no evidence to show that the DHA/lutein combination could have any positive effect on subjects already experiencing cognitive impairment.

WO2019/110591 discloses the use of a composition comprising lutein, zeaxanthin, meso-zeaxanthin, and at least one omega-3 fatty acid for use in the treatment of dementia in a human subject. Specifically, the data presented in the document show that a group of patients with Alzheimer's disease, when given a dietary supplement containing the composition, were more likely to demonstrate an improvement in cognitive function or to demonstrate less deterioration in cognitive function, compared to an age-matched control group of Alzheimer's disease patients who were not given the dietary supplement.

There are several systems commercially available for testing of cognitive function (Zygouris & Tsolak, 2015).

One method of assessing cognitive function is to use the RBANS tests (“Repeatable Battery for the Assessment of Neuropsychological Status”). The RBANS protocol tests five domains of cognition: immediate memory, visuospatial ability, language, attention, and delayed memory, using 12 sub-tests. The RBANS takes about 30 minutes to administer and is a core diagnostic tool for detecting and characterising dementia (Randolph et al., 1998 “The Repeatable Battery for the Assessment of Neuropsychological Status (RBANS): preliminary clinical validity” J Clin Exp NeuropsychoL 20(3) 310-319).

A computerised system is the CANTAB Connect Research software, available from Cambridge Cognition (www.cambricecognition.com; Tunbridge Court, Bottisham, Cambridge CB25 9TU, UK; and Cambridge Innovation Center, One Broadway, Cambridge, Mass. 02142, USA). This system comprises a battery of tests which are intended to target various specific domains of cognition: comprehension; executive function (working memory); reaction time; and episodic memory. The system includes four different tasks, which are referred to in the Cambridge Cognition publications (Cambridge Cognition 2016a, b) as MOT, SWM, RTI and PAL. These tests are designed to interrogate the four specific domains noted above. Thus MOT test targets comprehension, the SWM test interrogates working memory, the RTI test targets reaction time, and the PAL test interrogates episodic memory. The tests can be performed using a tablet computer with a touch screen, and require a finger-operated response.

The SWM (Spatial Working Memory task measures the ability to retain spatial information and manipulate it in working memory. SWM is a measure of the pre-frontal cortex brain region. In this task, the individual is presented with some boxes on the screen. The aim is to find the tokens that are hidden inside the boxes. Only one token will be hidden at a time.

Individuals must search for tokens by touching the coloured boxes (with the index finger of their dominant hand) to open them. The task becomes more difficult as the number of boxes (and tokens to find) increases. The key task instruction is that the tokens will never appear in the same box twice, so the individual must not return to a box if they have already found a token in that box. If the individual returns to a box where they have already found a token it will be classed as an error. If the individual first opens a box that is empty, they may return to that box again as a token could appear in that box at a later stage (e.g. the second or third time that they return to that box, depending on the difficulty level of the task). Importantly, when they first find a token in a box they must remember not to return to that box. Essentially, the individual must remember the location of the box in which they have or have not found a token during each level of the task.

The individual completes two practice rounds prior to commencing the SWM task. In each of the practice rounds, the individual must collect a total of three tokens. There are three levels of increasing difficulty for this task; stage 4 where the individual must locate four tokens in total—4 boxes appear on the screen at any one time, and only one of the 4 boxes will contain a token at a given time; stage 6 where the individual must locate six tokens in total (with 6 boxes on screen at any one time); and stage 8 where the individual must locate eight tokens in total (with 8 boxes on screen at any one time).

The task measures cognitive function by calculating the number of errors made by an individual in performing the task. The software recognises several categories of error:
A) “Between Errors”—these are defined as times the subject revisits a box in which a token has previously been found. The number of “between errors” can be calculated s for each of the three individual stages, and also for the combined total of “between errors” (i.e. between errors at stage 4, 6 and 8 added together).
B) “Within Errors”—these are the number of times a subject revisits a box already found to be empty during the same search.
C) “Double Errors”—the number of times a subject commits an error that is both a “between error” and a “within error”.
D) “Total Errors”—this is calculated by “Between Errors”+“Within Errors”—Double Errors.

Again the “Total Errors” can be calculated for each individual stage (4, 6 or 8), or a combined total for all three stages.

Other computer-based systems for measuring cognitive function are available and are reviewed by Zygouris & Tsolaki (cited elsewhere) and include, for example: CANS-MCI (Computer-Administered Neuropsychological Screen for Mild Cognitive Impairment) and CAMCI (Computer Assessment of Mild Cognitive Impairment, available from Psychology Software Tools, Inc. Pittsburgh, Pa. 15215, USA).

The content of all citations is incorporated herein by reference.

### SUMMARY OF THE INVENTION

In a first aspect the invention provides a method of improving the cognitive function of a cognitively healthy human adult, the method comprising administering to the human adult an effective amount of a composition comprising lutein, zeaxanthin, meso-zeaxanthin and at least one omega-3 fatty acid, over a period of at least 12 months, preferably at least 24 months, the improved cognitive function being a detectable improvement in: spatial working memory as measured by the CANTAB Connect Research software SWM task; and/or reduced reaction time as measured by the CANTAB Connect Research Software RTI task.

For present purposes, a human adult is considered “cognitively healthy” if they fulfil the following criteria: no self or family member reported memory loss; no rapidly progressive or fluctuating symptoms of memory loss; no established diagnosis of early dementia; and a score of 26 or higher in the Montreal Cognitive Assessment (MoCA) version 7.1 (Nasreddine et al., 2005; Luis, Keegan & Mullin, 2009).

Preferably a cognitively healthy human adult will also have an “immediate memory score” of 100 or more, as measured by the RBANS test (Randolph et al., 1998, cited above).

Preferably the method is performed on a mature subject (i.e. 65 years old or more).

In a second aspect, the invention provides a composition comprising lutein, zeaxanthin and meso-zeaxanthin and at least one omega-3 fatty acid, for use in improving the is cognitive function of a cognitively healthy human adult, the improvement in cognitive function being a detectable improvement in spatial working memory, as measured by the CANTAB Connect Research software SWM task; and/or reduced reaction time as measured by the CANTAB Connect Research Software RTI task.

The CANTAB Connect Research Spatial Working Memory (SWM) test requires retention and manipulation of visuospatial information. This self-ordered test has notable executive function demands and allows measurement of working memory errors.

The inventors have found that, in particular, the cognitive function of subjects is improved compared to control subjects when the difficulty of the task is increased. Thus, for example, at stage 6 and, more especially, at stage 8 of the SWM task, subjects who had been consuming the nutritional supplement in accordance with the invention made statistically significantly fewer errors than control subjects.

For the purposes of the present application, an improvement in spatial working memory is preferably detected by a reduction in the number of “Total Errors” made by the subject at stage 8 (“TE8”), or across all stages (“TE 4-8”) of the SWM test taken following consumption of the composition over the defined period of at least 12 months, preferably at least 24 months, (the test being taken within one week of the end of the defined period), compared to the total number of errors made by the subject across all the stages of the SWM test taken prior to commencing consumption of the composition. Preferably the total number of errors is reduced by 2 or more, more preferably by an amount in the range 3-5. Alternatively, the total number of errors is preferably reduced by at least 15%, more preferably by at least 20%, and most preferably by an amount in the range 20-25%.

The CANTAB Connect Research Reaction Time (RTI) test allows assessment of motor and mental response speeds, as well as measurement of movement time, reaction time and response accuracy. In the test, the subject must select and hold a button at the bottom of a computer tablet screen. Circles are presented above (one circle for the simple mode; and five circles for the five-choice made). In each case, a yellow dot will appear in one of the circles, and the subject must react as soon as possible, releasing the button at the bottom of the screen, and selecting the circle in which the dot appeared.

Outcome measures are divided into reaction time and movement time for both the simple and five choice variants.

In the present invention, it is preferred that the parameter selected for measurement is the reaction time in the ‘simple’ format test, and a detectable improvement in the reaction time is preferably detected by a reduction in the simple reaction time of the subject in the RTI test taken following consumption of the composition over the defined period of at least 12 months (the test being taken within one week of the end of the defined period), compared to the simple reaction time of the subject in the RTI test taken prior to commencing consumption of the composition. Desirably the simple reaction time of the subject is reduced by at least 10 milliseconds, preferably by at least 15 milliseconds, and desirably by an amount in the range 15-20 milliseconds. Alternatively stated, the simple reaction time is preferably reduced by at least 3%, more preferably at least 4%, and most preferably by an amount in the range 4.5-5.5%.

Preferably, in the first and second aspects of the invention, consumption of the composition by the subject for the defined period results in a detectable performance improvement in both the SWM test and the RTI test.

In the first or second aspect of the invention, the composition may be administered by any suitable route to a human subject including, for example, intra-venous administration. Preferably however the composition is administered orally, and advantageously the composition is formulated so as to be suitable for oral administration.

As noted previously, the composition comprises at least one omega-3 fatty acid. For present purposes, unless the context dictates otherwise, the term “fatty acid” is intended also to encompass not only the free acid but also derivatives of fatty acids, such derivatives encompassing in particular esters, especially esters formed with glycerol (monoglycerides, diglycerides and, preferably, triglycerides), and salts. Preferred salts are those containing monocations, such as Na⁺, K⁺ or NH₄⁺. Most preferred salts are those comprising metallic monocations. The free fatty acid or the triglyceride is the most preferred form of the compound.

The omega-3 fatty acid component of the composition preferably comprises an omega-3 polyunsaturated fatty acid or derivative thereof; most preferably docosahexaenoic acid (DHA) or a derivative thereof. The composition may contain two or more omega-3 fatty acids. The composition may comprise eicosapentaenoic acid (EPA). In one embodiment the composition comprises both DHA and EPA.

A convenient source of omega-3 fatty acids is fish oil. Accordingly, in a preferred embodiment, the composition comprises fish oil. Since fish oil has quite a strong odour, it may be preferred to use deodorized fish oil, which is commercially available. Another source of omega-3 fatty acids is nut oil. Without being bound by any particular theory, the inventors believe that DHA is the most active omega-3 fatty acid in terms of preventing and/or treating dementia. Nut oil does not contain substantial amounts of DHA and therefore is not preferred for the purposes of the present invention.

Other sources of fatty acids include algae (see Ji et al, 2015 “Omega-3 Biotechnology: A green and sustainable process for omega-3 fatty acids production” Front Bioeng. Biotechnol. 3, 158).

The composition may be formulated in a ‘bulk’ form, to be admixed, for example, with a conventional foodstuff, including dairy foodstuffs (e.g. incorporated into butter or ice cream) or non-diary foodstuffs (e.g. margarine, vegetable stock or fish-stock preparations). More preferably however the composition is formulated in unit dosage form, preferably one suitable for oral consumption by a human subject, including a tablet, capsule, gel, liquid, powder or the like. The one or more macular carotenoids may be granulated, for example as microcapsules, before inclusion in the formulation.

Conveniently, but not necessarily, the composition may be packaged in a foil blister pack, of the sort known to those skilled in the art. Desirably one or two of the doses are taken each day, the amount of active agents in the doses being adjusted accordingly.

The composition may conveniently comprise conventional diluents, especially vegetable oils such as sunflower, safflower, corn oil and rape seed oils, excipients, bulking agents and the like which are well known to those skilled in the art. Such substances include, calcium and/or magnesium stearate, starch or modified starch.

Other conventional formulating agents may be present in the composition, including any one or more of the following non-exhaustive list: acidity regulators; anticaking agents (e.g. sodium aluminosilicate, calcium or magnesium carbonate, calcium silicate, sodium or potassium ferricyanide), antioxidants (e.g. vitamin E, vitamin C, polyphenols), colorings (e.g. artificial colorings such as FD&C Blue No. 1, Blue No. 2, Green No. 3, Red No. 40, Red No. 3, Yellow No. 5 and Yellow No. 6; and natural colorings such as caramel, annatto, cochineal, betanin, turmeric, saffron, paprika etc.); color retention agents; emulsifiers; flavours; flavour enhancers; preservatives; stabilizers; sweeteners and thickeners.

Other optional ingredients of the composition include vitamins and/or minerals. Preferably the composition comprises vitamin E. Preferably the composition comprises at least one B vitamin. Preferably the composition comprises vitamin E and at least one B vitamin.

Typically the composition is administered at least once a week, preferably at least twice a week, more preferably three times a week, and most preferably daily. In a typical embodiment at least one unit dosage form of the composition is taken on a daily basis. The person skilled in the art will appreciate that the frequency of consumption can be adjusted to take account of the concentration of active agents (the macular carotenoids, and omega-3 fatty acid), present in the formulation. The administration of the composition can be adjusted accordingly.

In order to obtain optimum effect, the composition should be administered, typically at least 2 or 3 times a week, and preferably daily, over a period of at least 6 months, preferably over a period of at least 12 months, more preferably over a period of at least 18 months and most preferably over a period of 24 months. If desired, the composition may be administered indefinitely, for as long as the subject is able to consume or otherwise receive the composition.

For present purposes the “active agents” in the composition are considered to be the macular carotenoids and the at least one omega-3 fatty acid.

The precise concentration of the active agents in the composition of the invention is not critical: a beneficial effect on the subject can be obtained by consuming larger doses of a composition comprising lower concentrations of active agents, or vice versa.

The composition includes three macular carotenoids, and their respective ratio in the composition is not thought to be critical and can vary quite widely. For example, the percentage of either MZ or lutein in the composition can range from 10% to 90% (of the macular carotenoid present in the composition). The percentage of zeaxanthin can typically range from about 5 to 45% (of the macular carotenoid present in the composition). One particular composition has an MZ:lutein:zeaxanthin ratio of 10:10:2 (or 45%, 45%, 10%), but this is not essential. Another typical composition has a ratio (MZ:L:Z) of 12:10:2.

One preferred composition for use in performance of the invention is in unit dosage form, with each unit dose comprising 2 mgs meso-zeaxanthin, 18 mgs lutein and 2 mgs zeaxanthin (i.e. an MZ:L:Z ratio of 2:18:2). The composition preferably further comprises fish oil as a source of omega-3 fatty acid. Desirably each unit dose comprises 0.5-1.0 gram of fish oil.

Another composition for use in performance of the invention is in unit dosage form, with each unit dose comprising 10 mgs meso-zeaxanthin, 10 mgs lutein, 2 mgs zeaxanthin and fish oil (preferably 1 gm of fish oil).

Another composition for use in performance of the invention is in unit dosage form with each unit dose comprising 15 mgs meso-zeaxanthin, 5 mgs lutein, 1 mg zeaxanthin and fish oil (preferably 1 gm fish oil).

These doses may conveniently be presented as capsules. As the resulting capsules are quite large, and possibly difficult to swallow for elderly subjects, the unit dose may, for example, be halved and the subject thus swallows two such capsules in order to obtain the desired dose of active agents.

In one particular embodiment, the composition substantially consists of lutein, zeaxanthin, meso-zeaxanthin and fish oil, surrounded by an inert capsule material. Suitable materials for use as a capsule material are well-known to those skilled in the art and include, inter alia, gelatin and cellulose-derived polymers such as hypromellose.

Desirably an average daily dose of the composition provides a total macular carotenoid content of up to, but not exceeding, 100 mg, preferably up to, but not exceeding, 75 mg, and most preferably up to, but not exceeding, 50 mg. Desirably the average daily dose of the composition provides a minimum total macular carotenoid content of at least 18 mg, more preferably at least 20 mg, and most preferably at least 22 mg. Such concentrations are known to be well-tolerated with substantially no adverse effects.

Advantageously, the average daily dose of the composition provides an amount of omega-3 fatty acid in the range 10 mg to 2 gms, more preferably in the range 20 mgs to 2 gms, and most preferably in the range 25 mgs to 1 gram.

The foregoing omega-3 fatty acid content may be provided entirely by DHA, or by a combination of two or more omega-3 fatty acids, one of which is preferably DHA. DHA desirably constitutes at least 50% of the omega-3 fatty acid content of the composition, preferably at least 55%, more preferably at least 60%, and most preferably at least 65% of the omega-3 fatty acid content of the composition.

### BRIEF DESCRIPTION OF THE FIGURES

The various aspects and features of the invention will now be further described by way of illustrative embodiment and with reference to the following drawing figures, in which:

FIG. 1 illustrates the structure of the macular carotenoids lutein (L), zeaxanthin (Z) and meso-zeaxanthin (MZ);

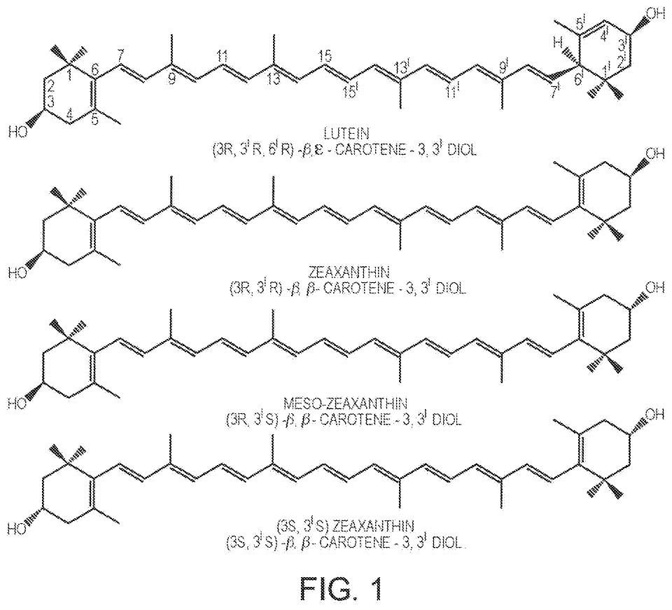

FIG. 2 is a graph showing the change in macular pigment optical volume (MPOV) over 24 months for active and placebo groups;

FIG. 3 is a graph showing the change in skin carotenoid score over 24 months for active and placebo groups;

FIG. 4 is a graph showing the change in serum lutein concentration over 24 months for active and placebo groups;

FIG. 5 is a graph showing the change in plasma DHA concentration over 24 months for active and placebo groups;

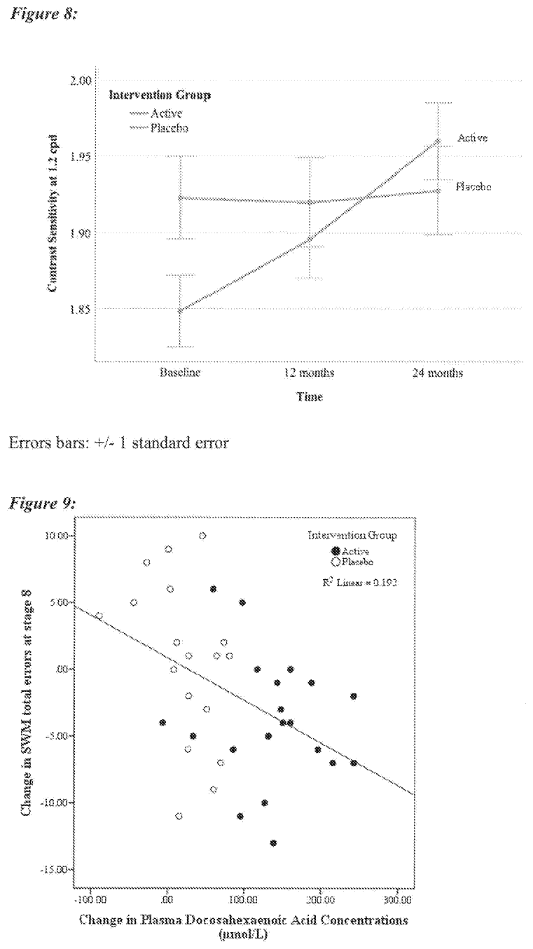

FIG. 6 is a graph showing the change in spatial working memory (SWM) total errors at stage 8 over 24 months for active and placebo groups;

FIG. 7 is a graph showing the change in SWM total errors across all stage over 24 months for active and placebo groups;

FIG. 8 is a graph showing the change in contrast sensitivity (CS) at 1.2 cpd over 24 months for active and placebo groups; and

FIGS. 9-16 are scatter plots illustrating the relationship between changes observed in various parameters.

### DETAILED DESCRIPTION OF THE INVENTION

### Examples

### Example 1—Cognitive Impairment Study (“CARES”)

The inventors organised a parallel group, double-blind, placebo-controlled randomised clinical trial to investigate the impact of nutritional supplementation on cognitive function in cognitively healthy, mature (≥65 years of age) adults.

### Carotenoid, Omega-3 Fatty Acid and Vitamin E Intervention in a Cognitively Healthy Sample

Concomitant with the growing population and increases in life expectancy in most regions is the increasing prevalence of age-related diseases. Dementia represents the most significant stage of cognitive decline and is one of the fastest growing age-related diseases. Estimates suggest that there are over 52 million adults worldwide with dementia, with prevalence numbers expected to double every 20 years (Alzheimer's Disease International, 2018). Dementia poses one of the greatest health care challenges to society in terms of personal, societal and financial implications. Current pharmacological interventions for AD, the most common form of dementia, are aimed at neurochemical imbalance and have limited success in stabilising or slowing the progression of the disease. Thus, emphasis is being placed on strategies to promote healthy ageing, with the aim of minimising the burden of disability and disease in later life and maximising the quality of life for individuals in their later years.

Accumulating evidence from observational and interventional studies suggests that good nutrition (e.g. fruits, vegetables and fish) can positively impact on cognition and AD risk. Advances in science and technology have increased our capacity to fully elucidate the unique neuroprotective mechanisms of specific nutrients that are likely to be driving the positive results that have been observed between good nutrition and healthy cognitive status. Given the abundance of ω-3 FAs in the brain, the selective presence of xanthophyll carotenoids and vitamin E in brain tissue, and their ability to independently and synergistically attenuate the mechanisms involved in the pathogenesis of AD (namely oxidative damage and inflammation), it is likely that they play a significant neuro-protective role by maintaining and optimising cognition and reducing the risk of cognitive s decline. However, well designed and appropriately powered interventional trials are needed to confirm this hypothesis. The Cognitive impAiRmEnt Study (CARES) was a parallel group, double-blind, placebo-controlled, randomised clinical trial studying two populations of interest. This trial (CARES Trial 1) investigated the impact of targeted nutritional supplementation on cognitive function in cognitively healthy older adults (≥65 years).

### Methodology

CARES trial 1 investigated the impact of 24-month supplementation with xanthophyll carotenoids, ω-3FAs and vitamin E on cognitive function in cognitively healthy older adults. Volunteers, primarily from the South-East of Ireland, were recruited through regional and national advertisement campaigns. Eligibility criteria for CARES included: aged ≥65 years; no self or family member reported memory loss; no rapidly progressive or fluctuating symptoms of memory loss; no established diagnosis of early dementia; no consumption of cognitive enhancement therapy such as cholinesterase inhibitors or N-methyl-D-aspartate receptor antagonists); no stroke disease (clinical stroke or stroke on CTB); no depression (under active review); no psychiatric illness (under active review of psychotropic medications); no glaucoma (acute angle); not consuming carotenoid or fish/cod liver oil supplements; and no fish allergy.

Prior to enrolment, all individuals completed a screening assessment to confirm eligibility. This included assessing cognitive function using the Repeatable Battery for the Assessment of Neuropsychological Status (RBANS) Record Form A and the Montreal Cognitive Assessment (MoCA) version 7.1. A description of each of these assessments is provided below. Individuals that fulfilled the criteria for each cognitive and functional assessment were invited to participate in the clinical trial. Individuals with borderline scores from the screening assessments were referred to a consensus panel (via email or conference call) consisting of two Consultant Geriatricians and a Clinical Neuropsychologist for assessment of eligibility (Albert et al., 2011; Dubois & Albert, 2004). Eligible individuals were then invited to enrol into the study. Prior to enrolment, written informed consent was obtained from all individuals. Ethical approval was granted by the Research Ethics Committees of the Waterford Institute of Technology and University Hospital Waterford in Waterford, Ireland. CARES (trial registration number. ISRCTNI0431469) adhered to the tenets of the declaration of Helsinki and followed the full code of ethics with respect to recruitment, testing and general data protection regulations as set out by the European Parliament and Council of the European Union.

Eligible individuals were randomised to either the active intervention group, which received a supplement containing 10 mg L; 10 mg MZ; 2 mg Z; 15 mg vitamin E (a-tocopherol); 1 g fish oil (of which 430 mg DHA and 90 mg EPA) or placebo (sunflower oil) intervention group. These doses were provided via two oval-size capsules. Individuals were instructed to consume two capsules per day with a meal. Frequent phone calls were made to ensure compliance. Tablet counting was also performed at follow-up. Study visits occurred at baseline, 12- and 24-months at a single site (Nutrition Research Centre Ireland [NRCI]). The trial commenced in March 2016 and concluded in June 2019 (i.e. last 24-month subject visit). Intervention randomisation was performed by an electronic trial management system (Trial Controller) designed by the inventors and their colleagues. This administration system was also used to document patient information (name and contact details), support the organisation and management of capsules required for the clinical trial and assist with the scheduling of study visits. The primary outcome measure of CARES was change in cognitive function. Secondary outcome measures (explained in greater detail below) included change in the following variables: macular pigment optical volume (MPOV); visual function; serum xanthophyll carotenoid concentrations; serum vitamin E concentrations; and plasma ω-3FA concentrations.

### Assessing Cognitive Unction

### Global Cognition

The MoCA was used at the screening stage to assess global cognition. It is a short (10 minute) cognitive screening tool with high sensitivity and specificity for detecting MCI (Luis, Keegan, & Mullan, 2009; Nasreddine et al., 2005). Thirty items assess multiple cognitive domains including visuospatial abilities, executive function, phonemic fluency, attention, immediate and delayed recall, language and orientation. In the present study, a score 226 was desirable for enrolment. The RBANS was used to measure global cognition at screening and at 12- and 24-month follow-up visits. Five domains of cognition (immediate memory, visuospatial ability, language, attention and delayed memory) were assessed using 12 sub-tests. The RBANS takes approximately 30 minutes to administer and is a core diagnostic tool for detecting and characterising dementia (Randolph, Tierney, Mohr, & Chase, 1998). In the present study, a score of ≥78 was desirable for enrolment.

### Specific Cognitive Domains

Specific domains of cognition were assessed using CANTAB Connect Research software (Cambridge Cognition, Cambridge, UK) (Zygouris & Tsolaki, 2015). This computerised software program was performed on an iPad and required a finger-operated response. The CANTAB protocol (Cambridge Cognition, 2016b) was followed in the administration of the test battery and was used to assess comprehension, executive function (working memory), reaction time and episodic memory (Cambridge Cognition, 2016a) at baseline and follow-up visits (see Table 1 for an overview of the CANTAB tests performed).

TABLE 1
Tasks performed in CARES to assess cognition using CANTAB

| | | | | Desirable | Performance |
|---|---|---|---|---|---|
| Cognitive domain | Task | Description | Outcome measure | score | ranges |
| Comprehension | MOT | Individuals must touch the flashing | Latency (speed of response) | Lower | — |
| | | cross shown in different | Total correct | Higher | 0-10 |
| | | locations on the screen. | Total errors | Lower | 0-10 |
| Executive function | SWM | The aim of this test is that, by touching | Between errors | Lower | 0-90 |
| (working memory) | | the boxes and using a process of elimination, | Total errors | Lower | 0-90 |
| | | the individual should find one ‘token’ | Strategy | Lower | 2-14 |
| | | in each of the boxes and use them to | | | |
| | | fill up an empty column on the right- | | | |
| | | hand side of the screen. The key task | | | |
| | | instruction is that the computer will never | | | |
| | | hide a token in the same coloured box, | | | |
| | | so once a token is found in a | | | |
| | | box the individual should not return | | | |
| | | to that box to look for another token. | | | |
| Reaction time | RTI | Individuals must press and hold down a | Simple reaction time | Lower | 100-5100 |
| | | touchscreen button at the bottom of the | Simple movement time | Lower | 100-5100 |
| | | screen. Circles are presented at the top of the | Simple error score | Lower | 0-30 |
| | | screen (one for simple mode, and five for | Five-choice reaction time | Lower | 100-5100 |
| | | the five-choice mode). In each case, a | Five-choice movement time | Lower | 100-5100 |
| | | yellow spot will appear in one of the circles. | Five-choice error score | Lower | 0-30 |
| | | Individuals must respond to the spot as | | | |
| | | quickly as they can by letting go of the | | | |
| | | button and touching inside the circle | | | |
| | | whore the yellow spot appeared. | | | |
| Episodic memory | PAL | Boxes are displayed on the screen and open | First attempt memory score | Higher | 0-20 |
| | | one by one in a randomized order to | No. patterns reached | Higher | 2-8 |
| | | reveal patterns hidden inside. The patterns | Total errors adjusted | Lower | 0-70 |
| | | are then displayed in the middle of the | | | |
| | | screen, one at a time, and the individual | | | |
| | | must touch the box where the pattern was | | | |
| | | originally located. If the individual | | | |
| | | makes an error, the patterns are | | | |
| | | re-presented to remind the | | | |
| | | individual of their locations. | | | |

MOT task Performance ranges: the minimum and maximum possible score for each measure; Latency: the speed (milliseconds) of response to the stimulus; Total correct: the number of correct responses; Total errors: the distance between the centre of the cross and the location touched; SWM task Between errors: the number of times the individual revisits a box in which a token has previously been found; Total errors: the number of times a box is selected that is certain not to contain a token and therefore should not have been visited by the individual; Strategy: for problems with six boxes or more. The number of distinct boxes used by the individual to begin a new search for a token, with the same problem; RTI task Simple reaction time: the duration between the onset of the stimulus and the time at which the individual released the button. Calculated for correct trials, where the stimulus could appear in one location only; Simple movement time: the time taken to touch the stimulus after the button has been released. Calculated for correct trials, where the stimulus could appear in one location only; Simple error score: the number of trials where the response status is any error (i.e. an inaccurate response, no response, or a premature response) for the assessment trial where stimuli appear on one location only. The error may be an inaccurate response, no response, or a premature response; Five-choice reaction time: the duration between the onset of the stimulus and the release of the button. Calculated for correct, assessed trials where the stimulus could appear in any of the five locations; Five-choice movement time: the time taken to touch the stimulus after the button has been released. Calculated for correct, assessed trials where the stimulus could appear in any of the five locations; Five-choice error score: the number of trials where the response status is any error (i.e. an inaccurate response, no response, or a premature response) for assessment trials where stimuli appear in any of five locations; PAL task First attempt memory score: the number of correct box choices that were made on the first attempt during assessment problems; No. patterns reached: the number of patterns on the last problem reached in the task; Total errors adjusted: the number of times the individual chose the incorrect box for a stimulus on assessment problems, plus an adjustment for the estimated no. errors they would have made on any problems, attempts and recalls they did not reach.

### Assessing Nutritional Status

### Macular Pigment

MP was measured by dual-wavelength autofluorescence (AF) using the Spectralis HRA+OCT MultiColor (Heidelberg Engineering GmbH, Heidelberg, Germany). Details of MP acquisition were described previously. In brief, pupillary dilation was performed prior to measurement and patient details were entered into the Heidelberg Eye Explorer (HEYEX version 1.7.1.0) software. Using the HEYEX software, the movie images were aligned and averaged, and a MP density map was created. MPOV, calculated as MP average times the area under the curve out to 7° eccentricity, is reported here.

### Skin Carotenoid Score

Carotenoid concentrations were also measured using the Pharmanex® BioPhotonic Scanner. This scanner measures carotenoid levels in human tissue at the skin surface using optical signals (resonant Raman spectroscopy). These signals identify the unique molecular structure of carotenoids, allowing their measurement without interference by other molecular substances. The individual was asked to place a specific point (between the maximal and distal palmar creases, directly below the fifth finger) of their right hand (previously cleaned with hand sanitiser) in front of the scanner's low-energy blue light for 30 seconds. From this, a skin carotenoid score was generated, which provided an indication of the individual's overall antioxidant levels. This was repeated twice more, and an average score was calculated. Based on this result an individual's score can be classified into three ranges; 0-29,000=low; 30,000-49,000=normal; 50,000+=high. This technology is safe and has been previously validated (Zidichouski, Mastaloudis, Poole, Reading, & Smidt, 2009).

### Biochemical Analysis of Serum Xanthophyll Carotenoids and Vitamin E

### High Performance Liquid Chromatography Materials

The carotenoid standards L, Z, Z racemic mixture and b-cryptoxanthin were purchased from CaroteneNature (Lupsingen, Switzerland). DL-α-tocopherol was purchased to Merck Serono Ltd (Dublin, Ireland). Butylated hydroxyltoluene (BHT), α-tocopheryl acetate and HPLC grade heptane were purchased from Sigma-Aldrich (Arklow, Ireland). HPLC grade methyl tert-butyl ether (MTBE), water and isopropanol were supplied by Fisher Scientific (Dublin, Ireland). HPLC grade hexane and ethanol 96% were supplied by VWR (Dublin, Ireland). NIST SRM 968f was purchased to the National Institute of Standards and Technology (NIST, Gaithersburg, Md., US).

### Serum Extraction

Non-fasting blood samples were collected at each study visit by standard venepuncture techniques. SST II Advance blood collection tubes (8.5 ml) were inverted at least 5 times to ensure thorough mixing of the silica clot activator. The blood samples were left to clot for 30 minutes at room temperature and then centrifuged at room temperature at 725 g for 10 minutes in a Gruppe GC12 centrifuge (Desaga Sarstedt, UK) to separate the serum from the whole blood. Following centrifugation, serum was transferred to light-resistant microtubes and stored at circa −80° C. until extraction. Xanthophyll carotenoids and α-tocopherol were extracted from serum samples and analysed by HPLC as previously described (Meagher et al. 2013).

### Biochemical Analysis of Plasma Omega-3 Fatty Acids

### Gas Chromatography Materials and Calibration

HPLC solvents were used throughout the analysis. Methyl heneicosanoate, methyl docosanoate, methyl tricosanoate, EPA, DHA and the authentic Fatty acid methyl esters (FAME) standard Mixture ME 1220 were sourced from Larodan (Solna, Sweden). Methyl heneicosanoate, methyl docosanoate, methyl tricosanoate and EPA were used to prepare four calibration solutions with six concentrations in the range of 2.5-200 mg/L. Linearity was checked along the concentration range for each standard FAME, and the slope of each calibration line was used to obtain an average response factor (RF) of 0.157±0.012 mAU g L⁻¹. Methyl heneicosanoate was used as recovery standard, and methyl tricosanoate was used to estimate matrix effect.

### Plasma Extraction

Lithium heparin blood collection tubes (6 ml) were inverted 8-10 times to ensure thorough mixing and centrifuged at 4° C. at 3000 rpm for 20 minutes in a 3-18K centrifuge (Sigma, Germany) to separate red blood cells and plasma. The time of blood collection and time of separation did not exceed 2 hours. Following centrifugation, all samples were transferred to light-resistant microtubes and stored at circa −80° C. until the time of analysis. Plasma ω-3FA analysis was performed by gas chromatography. FAME were prepared as previously described (Benner et al., 2018). Briefly, 50 μl of plasma were spiked with 20 μl of 2 mg/mL methyl tricosanoate and saponified with 2 mL of freshly prepared methanolic KOH 0.4 M during 10 min with gentle vortexing at room temperature. The samples were extracted three times with 2 mL of hexane and the combined extracts were dried in a vacuum centrifuge. The pellets were esterified with 2 mL of freshly prepared 5% methanolic sulfuric acid (v/v) at 80° C. for 30 min in a thermo-block. The FAME produced were extracted three times with 2 mL of hexane and dried in the vacuum centrifuge. The samples were resuspended in 0.4 mL of hexane containing 0.1 mg/mL of methyl heneicosanoate and prepared for GC-FID analysis. Methyl tricosanoete and methyl heneicosanoate 0.1 mg/mL were injected in triplicate to assess recovery and matrix effect respectively.

### Assessing Visual Function

### Visual Acuity

Best-corrected visual acuity (BCVA) was measured using a computerised Log MAR Early Treatment Diabetic Retinopathy Study (ETDRS) test chart (Test Chart 2000 Xpert; Thomson Software Solutions, Hatfield, UK) using the Sloan optotypes. Individuals were asked to read aloud the letters on the chart at a distance of 4 m. At the first incompletely read line, the letters of the line were randomised three times and an average score was calculated. BCVA was recorded in visual acuity rating (VAR). The eye with the best visual acuity was chosen as the study eye for assessment. Where both eyes had the same BCVA, the right eye was chosen.

### Contrast Sensitivity

Letter contrast sensitivity (CS) was measured for the study eye only using the computerised ETDRS test chart (Test Chart 2000 PRO) at five different spatial frequencies (1.2, 2.4, 6.0, 9.6, 15.15 cycles per degree [cpd]), using the Sloan optotypes. Individuals were asked to read letter aloud while fixating on the chart at a distance of 4 m. The letter set was randomised during the test at each change of contrast. The percentage contrast of letter optotypes is decreased in 0.15 log CS steps until the lowest contrast value for which subjects see at least three letters is reached. This process is then repeated for the remaining spatial frequencies. Each letter has a nominal log CS value of 0.03. Missed letters at any contrast level are noted. The resultant log CS value for the subject at a particular spatial frequency is calculated by adding any extra letter(s) and/or subtracting missed letters from best log CS value corresponding to the lowest percentage contrast.

### Demographic, Health and Lifestyle Data

Demographic, health and lifestyle data, medical history and current medication use were recorded via questionnaire. Height and weight measurements were recorded to calculate BMI (kg/m²). Smoking status was classified into never (smoked <100 cigarettes in lifetime), past (smoked ≥100 cigarettes in lifetime and none in the past year) or current smoker (smoked ≥100 cigarettes in lifetime and at least 1 cigarette in the last year). Alcohol consumption was measured in unit intake per week. One unit of alcohol (10 ml) was the equivalent to one of the following: a single measure of spirits (ABV 37.5%); half a pint of average-strength (4%) lager; two-thirds of a 125 ml glass of average-strength (12%) wine; half a 175 ml glass of average-strength (12%) wine; a third of a 250 ml glass of average-strength (12%) wine. Colour fundus photographs were taken to assess the presence of ocular pathology (Zeiss Visucam 200, Carl Zeiss Meditec AG, Jena, Germany).

### Statistical Analysis

The statistical package IBM SPSS version 25 was used and the 5% significance level applied for all analyses. Given that data were normally distributed, results were expressed as means±SD for numeric data. Categorical data were expressed as percentages. Between-group differences (i.e. active versus placebo) were analysed using Independent Samples t-tests or Chi-square tests as appropriate. The Monte Carlo Exact test was used where the assumption of the Chi-square tests were not met. rANOVA (repeated measures Analysis Of Variance) was used to assess Time-Group interaction effects across 3 time points (i.e. baseline, 12- and 24-month follow-up) between the active and placebo intervention groups for cognitive function, nutritional status and visual function variables. In cases where rANOVA showed interesting trends, further statistical analyses were conducted using paired samples t-tests to examine statistical difference within groups across beginning (i.e. at baseline) and end time points (i.e. at 24 months) for active and placebo intervention groups. A general linear model was used to assess (for dependent variables serum L, Z, MZ, and MP volume) the potential impact of sex, smoking habits, and alcohol consumption on Time and Time-Group effects. Finally, Spearman's rank correlation coefficient was used to investigate potential relationships between the observed changes in both cognitive and visual function and the observed changes in tissues and blood concentrations.

### Power Analysis

A sample size of 60 subjects was determined to be suitable in this study (with 30 subjects consuming the active supplement and 30 consuming placebo). Subjects were randomly allocated between the intervention groups and a 5% level of significance was chosen (i.e. a 95% confidence level). Calculations were based on rANOVA between two time points (i.e. baseline and end of study). All tests were assumed to be two-sided. RBANS across all five cognitive domains was an outcome measure for CARES. As all domains were considered to be of equal significance, the average scores were used for power analysis. Based on data provided from baseline, the mean RBANS score was 106 and mean standard deviation was 12. Assuming a correlation of 0.7 for within-subject RBANS scores between baseline and end of study, a statistical power of approximately 96% is estimated for an effect size of 10.6 (10% of baseline RBANS score) and 79% for an effect size of 7.95 (7.5% of RBANS score).

### Results

### Baseline Results

Baseline data for demographic, health and lifestyle, nutritional status, cognitive function and visual function are presented in Tables 2.1, 2.2, 2.3, and 2.4. All baseline variables were statistically comparable between both groups (p>0.05, for all). Of note, individuals (active n=5; placebo n=4) were excluded from analysis of CS 15.15 cpd as they recorded a baseline VAR of >90.

TABLE 2.1
Demographic, health and lifestyle data of active
and placebo intervention groups

| | Active | Placebo | |
|---|---|---|---|
| Variable | (n = 30) | (n = 30) | Sig. |
| Age (years) | 69.03 ± 4.41 | 69.77 ± 3.74 | 0.490 |
| Sex (% female) | 56.7 | 70.0 | 0.422 |
| Education (years) | 16.47 ± 1.61 | 17.41 ± 2.69 | 0.105 |
| BMI (kg/m²) | 28.92 ± 5.10 | 27.11 ± 4.18 | 0.138 |
| Exercise (min/week) | 288.17 ± 308.23 | 313.67 ± 282.12 | 0.739 |
| Smoking (%) | | | 0.168 |
| Never | 40.0 | 66.7 | |
| Past | 50.0 | 30.0 | |
| Current | 10.0 | 3.3 | |
| Alcohol consumption | | | 0.105 |
| (% units/week) | | | |
| 0 units | 36.7 | 33.3 | |
| 1 unit | 10.0 | 3.3 | |
| 2-5 units | 16.7 | 40.0 | |
| 6-10 units | 16.7 | 20.0 | |
| >10 units | 20.0 | 3.3 | |
| Medications | 3.07 ± 2.55 | 3.27 ± 2.97 | 0.780 |

Data displayed are mean±SD for numeric data and percentages for categorical data. Education: age (years) completed formal education; BMI: body mass index; Medications: the number of prescribed medications consumed. Education data missing for 1 individual in the active intervention group.

TABLE 2.2
Nutritional data of active and placebo intervention groups at baseline

| | Active | Placebo | |
|---|---|---|---|
| Variable | (n = 30) | (n = 30) | Sig. |
| MPOV | 5325 ± 2206 | 5575 ± 2002 | 0.650 |
| Skin carotenoid score | 30,593 ± 8317 | 32,167 ± 11.354 | 0.557 |
| Serum lutein (μmol/L) | 0.158 ± 0.060 | 0.203 ± 0.156 | 0.178 |
| Serum zeaxanthin (μmol/L) | 0.052 ± 0.014 | 0.059 ± 0.027 | 0.175 |
| Serum meso-zeaxanthin | 0 | 0 | — |
| (μmol/L) | | | |
| Serum vitamin E (μmol/L) | 29.272 ± 5.576 | 28.280 ± 5.693 | 0.518 |
| Plasma DHA (μmol/L) | 191.692 ± 82.881 | 214.406 ± 51.511 | 0.214 |
| Plasma EPA (μmol/L) | 128.572 ± 68.841 | 127.327 ± 39.650 | 0.935 |
| FFQ LZ intake | 16.13 ± 11.69 | 18.40 ± 15.74 | 0.529 |
| FFQ omega intake | 1.33 ± 1.28 | 1.28 ± 1.12 | 0.875 |

Data displayed are mean±SD; MPOV: a volume of macular pigment calculated as macular pigment average times the area under the curve out to 7° eccentricity (measured using the Heidelberg Spectralis®); DHA: docosahexaenoic acid; EPA: eicosapentaenoic acid; Data missing in the active intervention group for the following variables: MPOV (n=1); skin carotenoid score (n=3); serum xanthophyll carotenoid and vitamin E concentrations (n=4); plasma DHA and EPA (n=3); serum xanthophyll carotenoid and vitamin E data missing for 1 individual in the placebo group.

TABLE 2.3
Cognitive function data of active and placebo intervention groups at baseline

| Variable | Active | Placebo | Sig. |
|---|---|---|---|
| Global cognition | | | |
| MoCA | 27.53 ± 1.76 | 27.03 ± 1.16 | 0.199 |
| RBANS immediate memory | 108.37 ± 1330 | 107.0 ± 13.38 | 0.693 |
| RBANS visuospatial | 112.73 ± 13.20 | 115.40 ± 14.16 | 0.454 |
| RBANS language | 102.30 ± 10.28 | 101.40 ± 9.18 | 0.722 |
| RBANS attention | 100.83 ± 9.75 | 99.30 ± 15.24 | 0.644 |
| RBANS delayed memory | 107.30 ± 9.91 | 106.13 ± 10.94 | 0.667 |
| RBANS total scale | 107.70 ± 11.41 | 108.03 ± 11.34 | 0.910 |
| 4 mountains test | 9.14 ± 2.59 | 7.85 ± 2.89 | 0.113 |
| Comprehension | | | |
| Latency | 941.81 ± 219.17 | 1013.22 ± 234.54 | 0.228 |
| Working memory | | | |
| Between errors | | | |
| Stage 4 | 0.70 ± 1.47 | 1.13 ± 1.81 | 0.313 |
| Stage 6 | 3.87 ± 3.56 | 5.27 ± 3.77 | 0.144 |
| Stage 8 | 12.0 ± 4.81 | 11.07 ± 3.18 | 0.391 |
| All stages | 16.07 ± 7.67 | 16.63 ± 6.67 | 0.764 |
| Total errors | | | |
| Stage 4 | 0.87 ± 2.27 | 1.20 ± 2.09 | 0.556 |
| Stage 6 | 4.0 ± 3.63 | 5.57 ± 4.20 | 0.128 |
| Stage 8 | 12.50 ± 5.23 | 11.28 ± 3.13 | 0.291 |
| All stages | 16.86 ± 8.02 | 17.07 ± 6.68 | 0.915 |
| Strategy | 8.45 ± 2.28 | 9.27 ± 2.29 | 0.174 |
| Reaction time | | | |
| Simple reaction time | 371.62 ± 52.74 | 371.52 ± 46.22 | 0.994 |
| Simple movement time | 267.72 ± 71.57 | 293.32 ± 82.22 | 0.203 |
| Simple error score | 0.97 ± 1.43 | 0.93 ± 1.02 | 0.917 |
| Five-choice reaction time | 416.89 ± 45.37 | 425.91 ± 48.61 | 0.461 |
| Five-choice movement time | 310.26 ± 66.89 | 329.51 ± 75.68 | 0.301 |
| Five-choice error score | 0.23 ± 0.57 | 0.60 ± 1.0 | 0.088 |
| Episodic memory | | | |
| First attempt memory score | 10.40 ± 3.88 | 10.07 ± 3.26 | 0.720 |
| Total errors adjusted stage 2 | 0.27 ± 0.69 | 0.13 ± 0.51 | 0.398 |
| Total errors adjusted stage 4 | 1.33 ± 2.26 | 1.87 ± 2.53 | 0.393 |
| Total errors adjusted stage 6 | 6.77 ± 5.48 | 6.80 ± 4.38 | 0.979 |
| Total errors adjusted stage 8 | 17.0 ± 9.56 | 14.90 ± 9.38 | 0.394 |
| Total errors adjusted all stages | 25.37 ± 15.42 | 23.70 ± 13.05 | 0.653 |

Data displayed are mean±SD; MoCA: Montreal Cognitive Assessment; RBANS: Repeatable Battery for the Assessment of Neuropsychological Status; Data missing in the active intervention group for the following variables: 4MT score (n=8); SWM between errors stage 8 score (n=2); SWM between errors total score (n=1); SWM total errors stage 8 score (n=2); SWM total errors across all stages (n=1); SWM strategy score (n=1); Data missing in the placebo group for the following variables: 4MT score (n=4); SWM between errors stage 8 score (n=1); SWM total errors stage 8 score (n=1).

TABLE 2.4
Visual function data of active and placebo intervention groups at baseline

| Variable | Active | Placebo | Sig. |
|---|---|---|---|
| VAR | 99.59 ± 7.09 | 99.40 ± 7.29 | 0.921 |
| CS 1.2 cpd | 1.86 ± 0.16 | 1.88 ± 0.15 | 0.522 |
| CS 2.4 cpd | 1.87 ± 0.19 | 1.88 ± 0.20 | 0.910 |
| CS 6 cpd | 1.59 ± 0.22 | 1.55 ± 0.30 | 0.566 |
| CS 9.6 cpd | 1.34 ± 0.26 | 1.28 ± 0.31 | 0.463 |
| CS 15.15 cpd | 1.02 ± 0.30 | 0.99 ± 0.31 | 0.734 |

Data displayed are mean±SD; VAR: visual acuity rating; CS: contrast sensitivity. Data missing in the active intervention group for the following variables: VAR (n=1); CS 1.2 cpd (n=1); CS 2.4 cpd (n=1); CS 6 cpd (n=1); 9.6 cpd (n=1).

### Longitudinal Data

### Attrition Rate

Of the 60 individuals enrolled at baseline, 9 were lost at follow-up. Among individuals in the active arm of the intervention trial, 2 were no longer interested in participating. Among individuals in the placebo arm of the intervention trial, 2 were no longer interested in participating, 2 developed early-stage age-related macular degeneration and 2 developed other health issues. One adverse event was recorded during the trial. One patient (female, aged 77 years at baseline) reported severe diarrhoea 4 weeks after commencing the trial. Of note, this patient was previously diagnosed with cancer of the rectum. Upon trial completion, details of the intervention code revealed that this subject was enrolled into the placebo arm of the intervention trial. Thus, an attrition rate of 15% was recorded for CARES.

### Change in Outcome Variables Over Time

Final visit (i.e. 24-month) data of 1 individual (male, aged 77 years at baseline) in the placebo arm of the intervention trial were removed prior to rANOVA analysis as MZ was detected in 24-month (but not 12-month) serum (0.186 μmol/L). The presence of MZ suggested carotenoid supplementation and was retrospectively confirmed via telephone with the individual.

### Change in Nutritional Status Oer Time

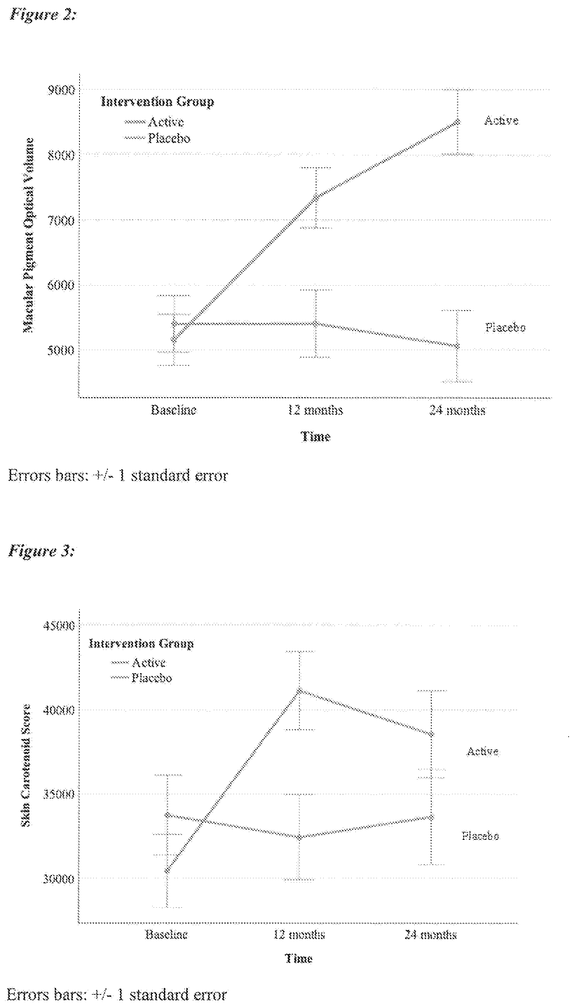

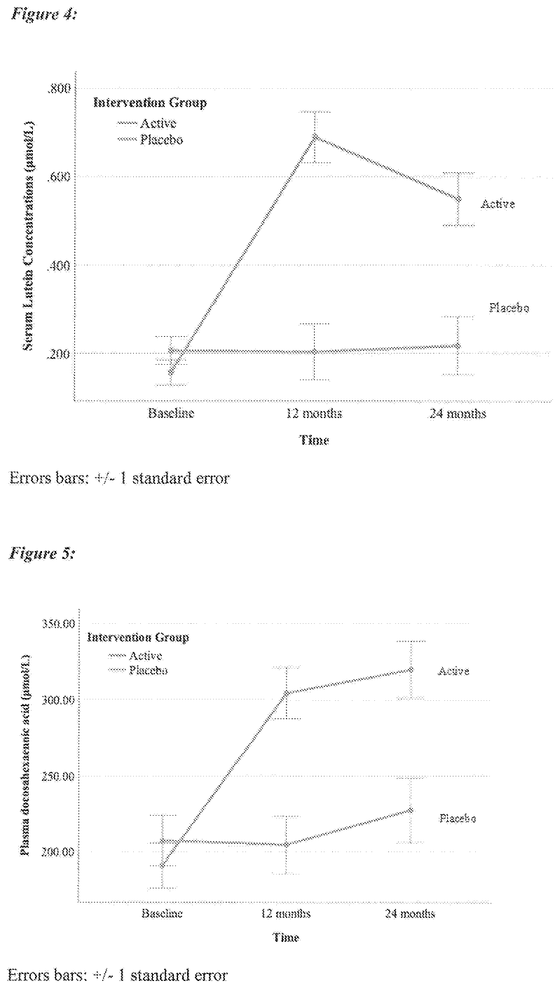

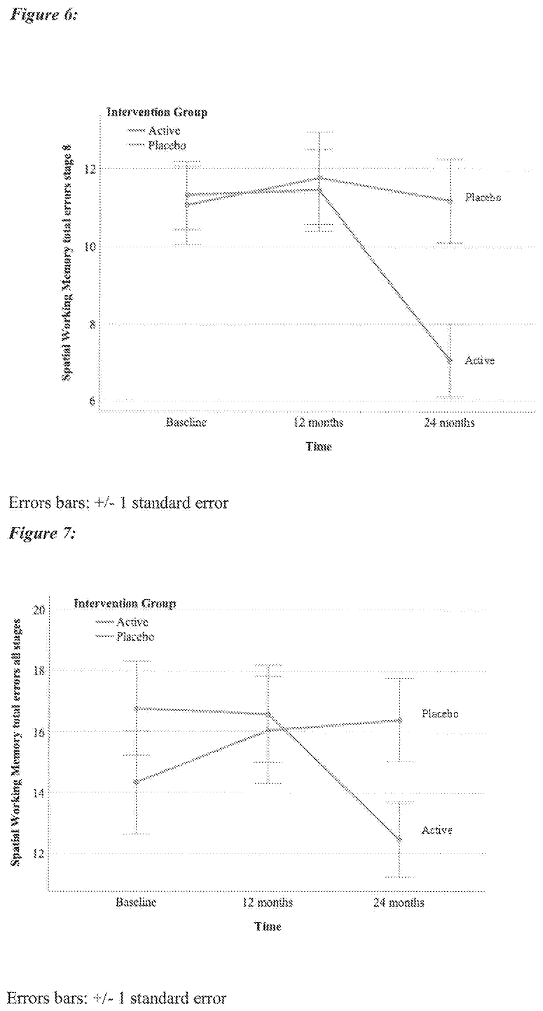

Table 3 (and FIGS. 2, 3 and 4 for example) summarise the observed changes in nutritional status in blood and tissue over the 24-month intervention period for active and placebo intervention groups. Statistically significant increases in MPOV and skin carotenoid score were observed among individuals consuming the active intervention in comparison to individuals receiving placebo (p<0.001, for both). Statistically significant increases were also observed for serum xanthophyll carotenoid concentrations and plasma ω-3FA concentrations in the active intervention group versus placebo (p<0.05, for all). Serum concentrations of vitamin E did not differ significantly between both groups (p>0.05).

### Effects of Demographic and Other Lifestyle Variables

The possibility of an interaction effect for sex, education, BMI, smoking status and alcohol consumption (units/week) on the statistically significant Time-Group effects observed for nutrition variables was examined using a general linear model. The dependent variables in these analyses included: MPOV; skin carotenoid score; serum L, Z MZ and vitamin E concentrations; and plasma DHA and EPA concentrations. No significant interactions were found (p>0.05, for all). Thus, for example, changes in MPOV did not differ by sex, BMI, smoking status nor alcohol consumption.

TABLE 3
Repeated measures analysis of variance of nutritional status variables at baseline, 12- and 24-months for active and placebo intervention groups

| | Baseline | | | | 12 months | | | |
|---|---|---|---|---|---|---|---|---|
| | Active | | Placebo | | Active | | Placebo | |
| Var. | n | M ± SD | n | M ± SD | M ± SD | % Δ; Outcome | M ± SD | % Δ; Outcome |
| MPOV | 26 | 5154 ± 2221 | 21 | 5399 ±1668 | 7338 ± 2704 | +42; Improved | 5403 ± 1847 | +0.01; Improved |
| SCS | 24 | 30,458 ± 8552 | 20 | 33,750 ± 12,615 | 41,125 ± 11,468 | +35; Improved | 32,250 ± 11,170 | −4; Declined |
| L | 23 | 0.157 ± 0.064 | 19 | 0.207 ± 0.190 | 0.689 ± 0.346 | +339; Improved | 0.204 ± 0.153 | −1; Declined |
| Z | 23 | 0.051 ± 0.014 | 19 | 0.064 ± 0.031 | 0.085 ± 0.035 | +67; Improved | 0.068 ± 0.042 | +6; Declined |
| MZ | 23 | 0 | 19 | 0 | 0.052 ± 0.032 | —; Improved | 0 | 0; Unchanged |
| Vit. E | 23 | 29.060 ± 5.715 | 19 | 28.646 ± 5.912 | 30.251 ± 5.557 | +4; Improved | 29.922 ± 6.810 | +4; Improved |
| DHA | 24 | 190.991 ± 85.894 | 19 | 207.415 ± 50.085 | 304.303 ± 95.382 | +59; Improved | 204.695 ± 61.975 | −1; Declined |
| EPA | 24 | 125.704 ± 67.679 | 19 | 116.687 ± 32.506 | 142.532 ± 54.126 | +13; Improved | 105.883 ± 41.300 | −9; Declined |

| | 24 months | | | | |
|---|---|---|---|---|---|
| | Active | | Placebo | | |
| Var. | M ± SD | % Δ: Outcome | M ± SD | % Δ; Outcome | T × G |
| MPOV | 8505 ± 2972 | +65; Improved | 5063 ± 1808 | −6; Declined | <0.001 |
| SCS | 38,542 ± 12,420 | +27; Improved | 33,650 ± 12,861 | −0.3; Declined | <0.001 |
| L | 0.550 ± 0.361 | +250; Improved | 0.218 ± 0.146 | +5; Improved | <0.001 |
| Z | 0.075 ± 0.033 | +47; Improved | 0.069 ± 0.032 | +8; Improved | 0.003 |
| MZ | 0.035 ± 0.031 | —; Improved | 0 | 0; Unchanged | <0.001 |
| Vit. E | 28.803 ± 5.399 | −1; Declined | 30.231 ± 7.217 | +6; Improved | 0.454 |
| DHA | 319.740 ± 111.854 | +67; Improved | 227.305 ± 58.274 | +10; Improved | <0.001 |
| EPA | 166.272 ± 77.310 | +32; Improved | 118.095 ± 36.092 | +1; Improved | 0.010 |

Data displayed are mean ± SD;
% Δ at 12 months: 12-month visit minus baseline visit expressed as a percentage;
% Δ at 24 months: 24-month visit minus baseline visit expressed as a percentage;
Outcome: Interpretation of direction of result (i.e. improved or declined over time);
T × G: Time × Group interaction effect;
MPOV: macular pigment optical volume, a volume of macular pigment calculated as macular pigment average times the area under the curve out to 7° eccentricity (measured using the Heidelberg Spectralis ®);
SCS: skin carotenoid score (measured using the Pharmanex BioPhotonic Scanner).
Serums lutein, zeaxanthin, meso-zeaxanthin carotenoid and vitamin E concentrations measured in μmol/L;
Plasma docosahexaenoic acid and eicosapentaenoic acid concentrations measured in μmol/L

### Change in Cognitive Function Over Time

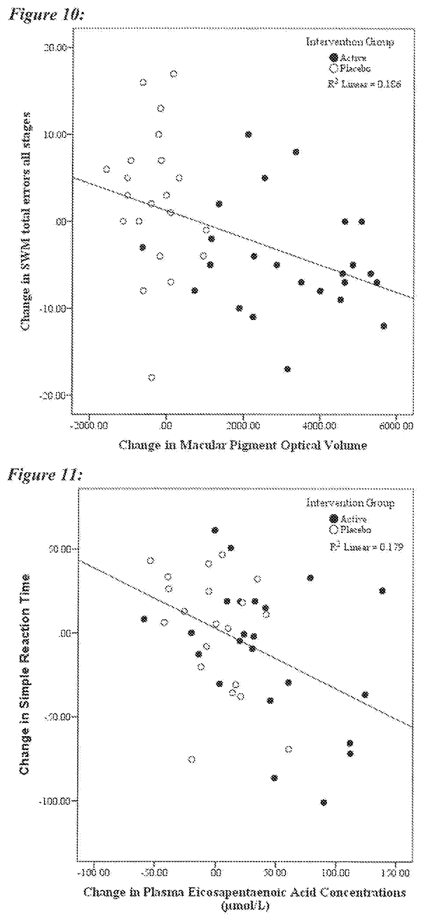

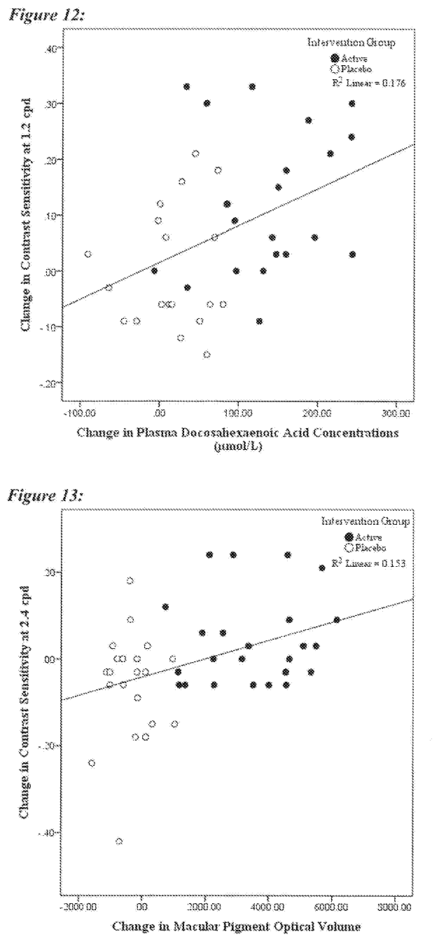

Table 4 and FIGS. 6 and 7 for example display the cognitive function variables for which a statistically significant Time-Group effect (p<0.05) was found (i.e. cognitive function variables for which the mean change, after 24 months, was significantly different between active and placebo groups). These included tasks assessing executive function (working memory), immediate memory and language. Individuals consuming the active intervention recorded significantly fewer errors (total at stage 8 and total errors across all stages) in the task assessing executive function in comparison to individuals in the placebo arm of the trial. Additionally, individuals in the active intervention group performed significantly better over time than the placebo group in the language domain of the RBANS (p<0.05).

TABLE 4
Repeated measures analysis of variance of cognitive function variables at
baseline, 12- and 24-months for active and placebo intervention groups

| | Baseline | | | | 12 months | | | |
|---|---|---|---|---|---|---|---|---|
| | Active | | Placebo | | Active | | Placebo | |
| Var. | n | M ± SD | n | M ± SD | M ± SD | % Δ; Outcome | M ± SD | % Δ; Outcome |
| SWM TE8 | 22 | 11.32 ± 4.67 | 17 | 11.06 ± 3.25 | 11.45 ± 5.31 | +1; Declined | 11.76 ± 4.37 | +6; Declined |
| SWM TE all stages | 22 | 16.77 ± 7.83 | 18 | 14.33 ± 6.37 | 16.59 ± 7.91 | −1; Improved | 16.06 ± 6.85 | +12; Declined |
| RBANS immediate | 28 | 109.64 ± 12.37 | 21 | 106.57 ± 14.89 | 110.46 ± 13.11 | +1; Improved | 110.67 ± 11.66 | +4; Improved |
| memory | | | | | | | | |
| RBANS language | 28 | 102.86 ± 10.27 | 21 | 101.33 ± 9.42 | 103.11 ± 9.80 | +0.2; Improved | 105.14 ± 9.22 | +4; Improved |

| | 24 months | | | | |
|---|---|---|---|---|---|
| | Active | | Placebo | | |
| Var. | M ± SD | % Δ; Outcome | M ± SD | % Δ; Outcome | T × G |
| SWM TE8 | 7.05 ± 4.13 | −38; Improved | 11.18 ± 4.83 | +1; Declined | 0.030 |
| SWM TE all stages | 12.45 ± 6.06 | −26; Improved | 16.39 ± 5.38 | +14; Declined | 0.015 |
| RBANS immediate | 113.07 ± 12.23 | +3; Improved | 119.10 ± 10.66 | +12; Improved | 0.017 |
| memory | | | | | |
| RBANS language | 110.82 ± 10.82 | +8; Improved | 105.52 ± 10.56 | +4; Improved | 0.009 |

Data displayed are mean±SD; % Δ at 12 months: 12-month visit minus baseline visit expressed as a percentage; % Δ at 24 months: 24-month visit minus baseline visit expressed as a percentage; Outcome: Interpretation of direction of result (i.e. improved or declined over time); T×G: Time×Group interaction effect; SWM TE8: spatial working memory total errors at stage 8, the number of times a box is selected that is certain not to contain a token and therefore should not have been visited by the individual at stage 8 of the assessment; SWM TE all stages: spatial working memory total errors across all stages, the number of times a box is selected that is certain not to contain a token and therefore should not have been visited by the individual, calculated across all stages of the assessment; RBANS: Repeatable Battery for the Assessment of Neuropsychological Status.

As noted previously, further statistical analyses were conducted in cases where rANOVA showed interesting trends. Paired samples t-tests were used to test statistical difference within groups across beginning and end time points for active and placebo intervention groups. Statistically significant improvements were observed among individuals in the active intervention group for executive function (between errors at stage 8 and total errors at stage 6) and simple reaction time (p<0.05, for all). Statistically significant improvements were observed in the RBANS total scale score for both groups (p=0.048 and p<0.001 for active and placebo groups, respectively) (see Table 5).

TABLE 5
Paired samples t-test of cognitive function variables at baseline and exit visits for active and placebo intervention groups

| | Active Intervention | | | | | | Placebo Intervention | | | | | |
|---|---|---|---|---|---|---|---|---|---|---|---|---|
| | | Baseline | 24 months | | | | | Baseline | 24 months | | | |
| Variable | n | M ± SD | M ± SD | % Δ | outcome | Sig. | n | M ± SD | M ± SD | % Δ | Outcome | Sig. |
| SWM between | 25 | 11.96 ± 484 | 9.24 ± 4.37 | −23 | Improved | 0.010 | 20 | 10.70 ± 3.47 | 11.20 ± 4.47 | +5 | Declined | 0.707 |
| errors stage 8 | | | | | | | | | | | | |
| SWM total | 27 | 4.33 ± 3.67 | 2.30 ± 2.23 | −47 | Improved | 0.007 | 22 | 4.64 ± 4.38 | 5.27 ± 3.38 | +14 | Declined | 0.576 |
| errors stage 6 | | | | | | | | | | | | |
| Simple | 28 | 370.17 ± 53.89 | 351.07 ± 37.83 | −5 | Improved | 0.031 | 22 | 368.17 ± 48.61 | 369.10 ± 44.85 | +0.3 | Declined | 0.900 |
| reaction time | | | | | | | | | | | | |
| RBANS total | 28 | 108.18 ± 11.64 | 111.71 ± 12.68 | +3 | Improved | 0.048 | 22 | 109.82 ± 11.15 | 116.64 ± 11.62 | +6 | Improved | <0.001 |
| scale score | | | | | | | | | | | | |

Data displayed are mean±SD; % Δ; 24-month visit minus baseline visit expressed as a percentage; SWM: spatial working memory; Simple reaction time: the duration between the onset of the stimulus and the time at which the individual released the button. Calculated for correct trials, where the stimulus could appear in one location only; Between errors: the number of times the individual revisits a box in which a token has previously been found; Total errors: the number of times a box is selected that is certain not to contain a token and therefore should not have been visited by the individual; RBANS: Repeatable Battery for the Assessment of Neuropsychological Status.

### Change in Visual Function Over Time

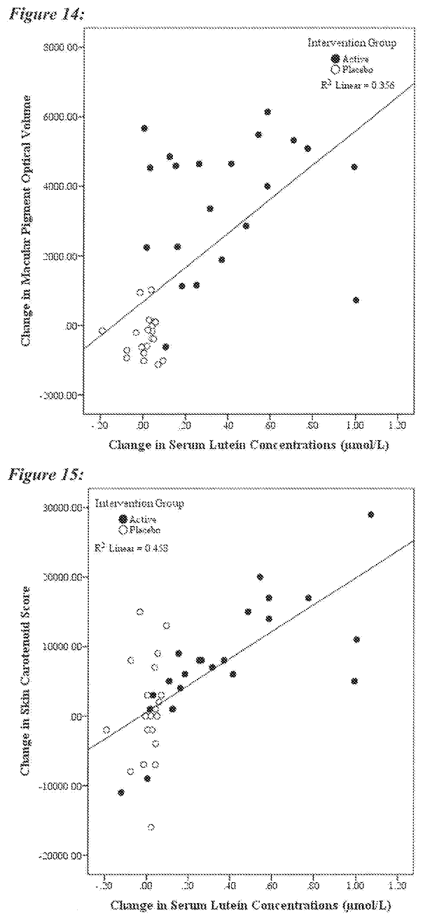

Over the 24-month intervention period, contrast sensitivity at 1.2 cpd and 2.4 cpd improved significantly (p<0.05, for both) in the active intervention group and was unchanged or declined in individuals consuming placebo (see Table 6 and FIG. 8, for example). Paired samples t-tests illustrated statistically significant (p=0.032) improvements in CS at 6 cpd among individuals in the active intervention group (i.e. an average improvement of 2 letters). A statistically significant (p=0.034) decline in CS at 2.4 cpd was observed among individuals receiving placebo (see Table 7).

TABLE 6
Repeated measures analysis of variance of visual function variables at
baseline, 12- and 24-months for active and placebo intervention groups

| | Baseline | | | | 12 months | | | |
|---|---|---|---|---|---|---|---|---|
| | Active | | Placebo | | Active | | Placebo | |
| Variable | n | M ± SD | n | M ± SD | M ± SD | % Δ; Outcome | M ± SD | % Δ; Outcome |
| VAR | 26 | 99.62 ± 7.16 | 20 | 99.30 ± 7.90 | 98.19 ± 6.70 | −1; Declined | 97.10 ± 11.12 | −2; Declined |
| CS 1.2 cpd | 26 | 1.85 ± 0.13 | 20 | 1.92 ± 0.11 | 1.90 ± 0.15 | +3; Improved | 1.92 ± 0.10 | 0; Unchanged |
| CS 2.4 cpd | 26 | 1.87 ± 0.17 | 20 | 1.94 ± 0.13 | 1.88 ± 0.19 | −1; Declined | 1.87 ± 0.17 | −4; Declined |
| CS 6 cpd | 26 | 1.59 ± 0.22 | 20 | 1.61 ± 0.23 | 1.56 ± 0.22 | −2; Declined | 1.54 ± 0.24 | −4; Declined |
| CS 9.6 cpd | 26 | 1.33 ± 0.25 | 20 | 1.34 ± 0.29 | 1.30 ± 0.26 | −2; Declined | 1.33 ± 0.26 | −1; Declined |
| CS 15.15 cpd | 26 | 1.03 ± 0.28 | 18 | 0.98 ± 0.32 | 0.97 ± 0.28 | −6; Declined | 0.97 ± 0.32 | −1; Declined |

| | 24 months | | | | |
|---|---|---|---|---|---|
| | Active | | Placebo | | |
| Variable | M ± SD | % Δ; Outcome | M ± SD | % Δ; Outcome | T × G |
| VAR | 98.42 ± 5.26 | −1; Declined | 97.15 ± 9.66 | −2; Declined | 0.802 |
| CS 1.2 cpd | 1.96 ± 0.13 | +6; Improved | 1.93 ± 0.13 | +1; Improved | 0.018 |
| CS 2.4 cpd | 1.91 ± 0.17 | +2; Improved | 1.88 ± 0.17 | −3; Declined | 0.023 |
| CS 6 cpd | 1.66 ± 0.19 | +4; Improved | 1.58 ± 0.27 | −2; Declined | 0.099 |
| CS 9.6 cpd | 1.35 ± 0.21 | +2; Improved | 1.29 ± 0.31 | −4; Declined | 0.242 |
| CS 15.15 cpd | 1.01 ± 0.28 | −2; Declined | 0.94 ± 0.35 | −4; Declined | 0.565 |

Data displayed are mean±SD; % Δ at 12 months: 12-month visit minus baseline visit expressed as a percentage; % Δ at 24 months: 24-month visit minus baseline visit expressed as a percentage; Outcome: Interpretation of direction of result (i.e. improved or declined over time); T×G: Time×Group interaction effect; VAR: visual acuity rating; CS: contrast sensitivity expressed in cycles per degree.

TABLE 7
Paired samples t-test of visual function variables at baseline and exit visits for active and placebo intervention groups

| | | Baseline | 24 months | | | | | Baseline | 24 months | | | |
|---|---|---|---|---|---|---|---|---|---|---|---|---|
| Variable | n | M ± SD | M ± SD | % Δ | Outcome | Sig. | n | M ± SD | M ± SD | % Δ | Outcome | Sig. |
| VAR | 26 | 99.62 ± 7.16 | 98.42 ± 5.26 | −1 | Declined | 0.234 | 21 | 99.62 ± 7.83 | 97.57 ± 9.62 | −2 | Declined | 0.048 |
| CS 1.2 cpd | 26 | 1.85 ± 0.13 | 1.96 ± 0.13 | +6 | Improved | <0.001 | 21 | 1.93 ± 0.11 | 1.93 ± 0.13 | 0 | Unchanged | 0.983 |
| CS 2.4 cpd | 26 | 1.87 ± 0.17 | 1.91 ± 0.17 | +2 | Improved | 0.053 | 21 | 1.95 ± 0.13 | 1.89 ± 0.17 | −3 | Declined | 0.034 |
| CS 6 cpd | 26 | 1.59 ± 0.22 | 1.66 ± 0.19 | +4 | Improved | 0.032 | 21 | 1.63 ± 0.23 | 1.59 ± 0.27 | −3 | Declined | 0.259 |
| CS 9.6 cpd | 26 | 1.33 ± 0.25 | 1.35 ± 0.21 | +2 | Improved | 0.649 | 21 | 1.35 ± 0.29 | 1.30 ± 0.30 | −4 | Declined | 0.087 |
| CS 15.15 cpd | 22 | 1.03 ± 0.28 | 1.01 ± 0.28 | −2 | Declined | 0.788 | 19 | 1.00 ± 0.33 | 0.95 ± 0.34 | −5 | Declined | 0.450 |

Data displayed are mean±SD; % Δ; 24-month visit minus baseline visit expressed as a percentage; VAR: visual acuity rating; CS: Contrast sensitivity expressed in cycles per degree.

### Relationships Between Change in Nutrition Status and Change in Cognitive Function

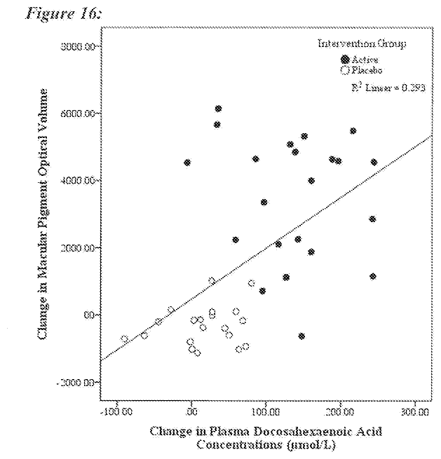

Spearman's rank correlation coefficient was used to investigate whether or not the observed changes in cognitive scores were related to the observed changes in nutritional status (see Table 8 and FIGS. 9, 10 and 11). Of interest, the observed reduction in the number of total errors made during the SWM (executive function) task at stage 8 and across all stages were inversely and significantly related to the observed increases in MPOV, serum L and MZ levels and plasmas DHA and EPA concentration (p<0.05, for all). In addition, the observed improvement in reaction time (attention) task was inversely and significantly related to the observed increase in MPOV and plasma concentrations of EPA (p<0.05, for both).

TABLE 8
Spearman's rank correlation coefficient between observed changes in
nutritional status and observed changes in cognitive function variables.

| | Observed change in | | | Observed change in | | | | | |
|---|---|---|---|---|---|---|---|---|---|
| | SWM total errors at | | | SWM total errors across | | | Observed change in | | |
| Observed change in | stage 8 | | | all stages | | | simple reaction time | | |
| nutritional status | R | P | N | R | P | N | R | P | N |
| MPOV | −0.452 | 0.002 | 45 | −0.458 | 0.002 | 45 | −0.353 | 0.014 | 48 |
| Serum L | −0.378 | 0.019 | 38 | −0.330 | 0.040 | 39 | −0.221 | 0.154 | 43 |
| Serum MZ | −0.411 | 0.010 | 38 | −0.435 | 0.006 | 39 | −0.247 | 0.110 | 43 |
| Plasma DHA | −0.446 | 0.005 | 38 | −0.408 | 0.010 | 39 | −0.156 | 0.316 | 43 |
| Plasma EPA | −0.310 | 0.058 | 38 | −0.317 | 0.050 | 39 | −0.365 | 0.016 | 43 |

Observed change, exit visit data minus baseline visit data; MPOV, a volume of macular pigment calculated as macular pigment average times the area under the curve out to 7° eccentricity (measured using the Heidelberg Spectralis®); Serums lutein, zeaxanthin, meso-zeaxanthin carotenoid and vitamin E concentrations measured in μmol/L; Plasma docosahexaenoic acid and eicosapentaenoic acid concentrations measured in μmol/L; Total errors: the number of times a box is selected that is certain not to contain a token and therefore should not have been visited by the individual; Simple reaction time: the duration between the onset of the stimulus and the time at which the individual released the button. Calculated for correct trials, where the stimulus could appear in one location only.

### Relationship Between Change in Nutritional Status and Change in Visual Function

The observed changes in CS at 1.2 cpd was positively and significantly related to the observed changes in serum L, serum MZ and plasma DHA concentrations (p<0.05, for all) (i.e. individuals with higher concentrations of these nutrients in blood had significantly better CS at 1.2 cpd than individuals with lower concentrations of these nutrients). Additionally, the observed change in CS at 9.6 cpd was also positively and significantly related to the observed change in MPOV and serum MZ concentrations (p<0.05, for both) (see Table 9 and FIGS. 12 and 13).

TABLE 9
Spearman's rank correlation coefficient between
observed changes in nutritional status and observed
changes in visual function variables.

| | Observed change in | | | Observed change in | | |
|---|---|---|---|---|---|---|
| Observed change in | CS 1.2 cpd | | | CS 2.4 cpd | | |
| nutritional status | R | P | N | R | P | N |
| MPOV | 0.278 | 0.061 | 46 | 0.325 | 0.027 | 46 |
| Serum L | 0.319 | 0.045 | 40 | 0.267 | 0.095 | 40 |
| Serum MZ | 0.352 | 0.026 | 40 | 0.369 | 0.019 | 40 |
| Plasma DHA | 0.417 | 0.007 | 41 | 0.257 | 0.104 | 41 |

Observed change, exit visit data minus baseline visit data; CS: contrast sensitivity; MPOV, a volume of macular pigment calculated as macular pigment average times the area under the curve out to 7° eccentricity (measured using the Heidelberg Spectralis®); Serum lutein and meso-zeaxanthin and plasma docosahexaenoic acid concentrations measured in μmol/L.

### Tissue and Blood Response to Nutritional Supplementation

The observed change in serum carotenoid concentrations (i.e. L, Z and MZ) were all, independently, positively and significantly related to the observed increases in MPOV and skin carotenoid concentrations (p<0.05, for all). Similarly, the observed change in plasma concentrations of DHA and EPA were both positively and significantly related to the observed change in MPOV and skin carotenoid concentrations (p<0.05, for both). Overall, the observed biochemical changes were positively and significantly related to the observed changes in tissue. Moreover, the observed changes in plasma concentrations of omega-3 fatty acids (DHA and EPA) were positively and significantly related to the observed changes in serum xanthophyll concentrations (L, Z and MZ) (p<0.05, for all). See Table 10 and FIGS. 14-16).

TABLE 10
Spearman's rank correlation coefficients between the observed changes in tissue
carotenoid concentrations and the observed change in biochemical variables.

| | Observed change in | | | Observed change in | | | Observed change in | | | Observed change in | | |
|---|---|---|---|---|---|---|---|---|---|---|---|---|
| Observed change in | MPOV | | | SCS | | | L | | | Z | | |
| nutritional status | r | p | n | r | p | n | r | p | n | r | p | n |
| MPOV | 1 | | | 0.393 | <0.001 | 45 | 0.617 | <0.001 | 41 | 0.373 | 0.016 | 41 |
| SCS | 0.393 | 0.008 | 45 | 1 | | | 6.704 | <0.001 | 42 | 0.596 | <0.001 | 42 |
| L | 0.617 | <0.001 | 41 | 0.704 | <0.001 | 42 | 1 | | | 0.810 | <0.001 | 43 |
| Z | 0.373 | 0.016 | 41 | 0.596 | <0.001 | 42 | 0.810 | <0.001 | 43 | 1 | | |
| MZ | 0.756 | <0.001 | 41 | 0.580 | <0.001 | 42 | 0.816 | <0.001 | 43 | 0.586 | <0.001 | 43 |
| Vitamin E | −0.240 | 0.130 | 41 | −0.108 | 0.494 | 42 | 0.068 | 0.666 | 43 | 0.126 | 0.421 | 43 |
| DHA | 0.566 | <0.001 | 42 | 0.490 | 0.001 | 42 | 0.711 | <0.001 | 42 | 0.487 | 0.001 | 42 |
| EPA | 0.465 | 0.002 | 42 | 0.242 | 0.123 | 42 | 0.578 | <0.001 | 42 | 0.419 | 0.006 | 42 |

| | Observed change in | | | Observed change in | | | Observed change in | | | Observed change in | | |
|---|---|---|---|---|---|---|---|---|---|---|---|---|
| Observed change in | MZ | | | vitamin E | | | DHA | | | EPA | | |
| nutritional status | r | p | n | r | p | n | r | p | n | r | p | n |
| MPOV | 0.756 | <0.001 | 41 | −0.240 | 0.130 | 41 | 0.566 | <0.001 | 42 | 0.465 | 0.002 | 42 |
| SCS | 0.580 | <0.001 | 42 | −0.108 | 0.494 | 42 | 0.490 | 0.001 | 42 | 0.242 | 0.123 | 42 |
| L | 0.816 | <0.001 | 42 | 0.068 | 0.666 | 43 | 0.711 | <0.001 | 42 | 0.578 | <0.001 | 42 |
| Z | 0.586 | <0.001 | 43 | 0.126 | 0.421 | 43 | 0.487 | 0.001 | 42 | 0.419 | 0.006 | 42 |

| | | | | | | | | | | |
|---|---|---|---|---|---|---|---|---|---|---|
| MZ | 1 | −0.139 | 0.373 | 43 | 0.723 | <0.001 | 42 | 0.494 | 0.001 | 42 |

| | | | | | | | | | | |
|---|---|---|---|---|---|---|---|---|---|---|
| Vitamin E | −0.139 | 0.373 | 43 | 1 | −0.030 | 0.849 | 42 | 0.105 | 0.508 | 42 |

| | | | | | | | | | | |
|---|---|---|---|---|---|---|---|---|---|---|
| DHA | 0.723 | <0.001 | 42 | −0.030 | 0.849 | 42 | 1 | 0.655 | <0.001 | 44 |

| | | | | | | | | | | |
|---|---|---|---|---|---|---|---|---|---|---|
| EPA | 0.494 | 0.001 | 42 | 0.105 | 0.508 | 42 | 0.655 | <0.001 | 44 | 1 |

Observed change, exit visit data minus baseline visit data; CS: contrast sensitivity; MPOV, a volume of macular pigment calculated as macular pigment average times the area under the curve out to 7° eccentricity (measured using the Heidelberg Spectralis®); SCS: skin carotenoid score (measured using the Pharmanex BioPhotonic Scanner). Serums lutein, zeaxanthin, meso-zeaxanthin carotenoid and vitamin E concentrations measured in μmol/L; Plasma docosahexaenoic acid and eicosapentaenoic acid concentrations measured in μmol/L.

### DISCUSSION

### Summary of Findings

Statistically significant improvements in nutritional status (in blood and tissue) were observed in individuals that consumed a combination of xanthophyll carotenoids, ω-3 FAs and vitamin E for 24 months. Individuals in the active intervention also exhibited statistically significant improvements in vision and cognition. Specifically, individuals consuming this nutritional combination exhibited statistically significant improvements in executive function (working memory), attention and some domains of global cognition in comparison to individuals consuming placebo. Moreover, the magnitude of change in nutritional status observed in both blood and tissue was directed related to the magnitude of change observed in cognitive and visual function.

With specific reference to measures of executive function, individuals in the active arm of the trial made significantly fewer errors at the final stage (stage 8) of the SWM test, and significantly fewer errors across all stages than individuals receiving placebo. Strikingly, the observed changes in blood and tissue concentrations of these particular nutrients were directly related to the observed improvements in this task.

In the present study, the encoding and retrieval of information was comparable between both active (errors at baseline=0.93; errors at final visit=0.86) and placebo groups (errors at baseline=0.80; errors at final visit=0.30) (p=0.797) during a working memory task with few stimuli (i.e. stage 4 of the SWM task where the individual had to locate 4 tokens). However, as the cognitive load increased (e.g. from 4 to 6 tokens), individuals in the active intervention (errors at baseline=4.50; errors at final visit=2.27) outperformed individuals in the placebo group (errors at baseline=4.26; errors at final visit=4.74) (p=0.072), with significantly better performance in the latter (i.e. stage 8) and total stages (p<0.05, for both) of the SWM task (see Table 4), where the cognitive load was at its highest. This suggests that the working memory capacity of individuals was favourably altered over time and that these positive changes may be attributed to the enrichment of specific nutrients in blood and tissue, given that the magnitude of change in nutritional status was related to the magnitude of change in cognitive function.

### Interaction Between Carotenoids and Omega-3 Fatty Acids

An additional and important finding from CARES Trial relates to the positive and significant relationships observed between tissue and blood concentrations of the specific nutrients tested in the intervention trial. The results suggest that the presence of ω-3 FAs in blood improved the biochemical response of the xanthophyll carotenoids, which in turn improved their respective concentrations in tissue. This improved delivery of these nutrients is likely to have contributed to the observed improvements in cognitive and visual function, thus supporting a plausible rationale for consumption of these nutrients in combination with one another for cognitive (and indeed ocular) health. The present study also relates the observed changes in blood and tissues to measures of function, primarily working memory and contrast sensitivity. It is possible that the interaction between xanthophyll carotenoids and ω-3 FAs is due to the hydrophobic nature of the xanthophylls, as it is known that consuming foods rich in carotenoids in the presence of oils or cholesterol can facilitate the uptake and bio-accessibility of carotenoids (van het Hof, et al., 2000).

### Non-Response to Vitamin E Supplementation

While individuals in the active intervention group responded to nutritional supplementation in terms of statistically significant improvements in serum xanthophyll carotenoid levels and plasma ω-3 FA levels, significant improvements were not observed for serum concentrations of α-tocopherol. Reasons underlying the poor vitamin E response following supplementation remain unclear. However, it is possible that circulating levels of vitamin E were sufficiently high at baseline and that additional amounts of vitamin E were not needed and thus excreted. In a national survey of almost 1,200 individuals aged 18+ years, circulating vitamin E concentrations in blood were reported as 32.04 μmol/L (approx. 13.8 mg) (Zhao et al., 2014). Given that a large amount of vitamin E is already present in blood in comparison to carotenoids for example, it is likely that the amount of α-tocopherol provided in the CARES supplement was insufficient to have any meaningful effect on circulating vitamin E levels in blood.

### CONCLUSION

In conclusion, the inventors have shown improvements in working memory, attention, domains of global cognition and measures of CS following 24-month supplementation with a combination of xanthophyll carotenoids (L, Z, MZ), ω-3 FAs (DHA, EPA) and vitamin E. The observed functional improvements were directly related to the observed increases of these nutrients (except vitamin E) in blood and tissue. The results suggest that the presence of ω-3 FAs in blood improved the biochemical response of the xanthophyll carotenoids, which in turn improved their respective concentrations in tissue and positively impacted on function. These results support a plausible rationale whereby these nutrients work together to optimise the neuro-cognitive environment. These findings have important implications for older adults in terms of enabling individuals to continue to function independently and interact with their environment in a meaningful way.

As the present invention may be embodied in several forms without departing from the spirit or essential characteristics thereof, it will be understood that the invention is not limited by the details of the foregoing description, unless otherwise specified, but rather should be construed broadly within its spirit and scope as defined in the appended claims, and therefore all changes and modifications that fall within the metes and bounds of the claims. Accordingly, the invention is defined by the appended claims.

### LIST OF REFERENCES

Adams, H. H., et al., (2015). Genetic risk of neurodegenerative diseases is associated with mild cognitive impairment and conversion to dementia. Alzheimers Dement, 11(11), 1277-1285. doi:10.1016/j.jalz.2014.12.008
Age-Related Eye Disease Study Research Group. (2001). A randomized, placebo-controlled, clinical trial of high-dose supplementation with vitamins C and E, beta carotene, and zinc for age-related macular degeneration and vision loss: AREDS report no. 8. Arch Ophthalmol, 119(10), 1417-1436.
Agrawal, R, & Gomez-Pinilla, F. (2012). ‘Metabolic syndrome’ in the brain: deficiency in omega-3 fatty acid exacerbates dysfunctions in insulin receptor signalling and cognition. The Journal of Physiology, 590(Pt 10), 2485-2499.
Akbar, M., et al., (2005). Docosahexaenoic acid: A positive modulator of Akt signaling in neuronal survival. Proceedings of the National Academy of Sciences of the United States of America, 102(31), 10858-10863. doi: 10.1073/pnas.0502903102
Akbaraly, N. T., et al., (2007). Plasma carotenoid levels and cognitive performance in an elderly population: results of the EVA Study. J Gerontol A Biol Sci Med Sci, 62(3), 308-316.
Akuffo, K. O., et al., (2015). Sustained supplementation and monitored response with differing carotenoid formulations in early age-related macular degeneration. Eye (Lond), 29(7), 902-912. doi:10.1038/eye.2015.64, Epub 2015 May 15.
Albert, M. S., et al., (2011). The diagnosis of mild cognitive impairment due to Alzheimer's disease: recommendations from the National Institute on Aging-Alzheimer's Association workgroups on diagnostic guidelines for Alzheimer's disease. Alzheimers. Dement., 7(3), 270-279.
Alcaino, J., Baeza, M., & Cifuentes, V. (2016). Carotenoid Distribution in Nature. Subcell Biochem, 79, 3-33. doi:10.1007/978-3-319-39126-7_1
Allen, T. A., & Fortin, N. J. (2013). The evolution of episodic memory. Proc Natl Acad Sci USA, 110 Suppl2, 10379-10386. doi: 10.1073/pnas. 1301199110
Alzheimer's Association. (2016). 2016 Alzheimer's disease facts and figures. Alzheimers Dement, 12(4), 459-509.
Alzheimer's Disease International. (2018). Dementia Statistics. Retrieved from https://www.alz.co.uk/research/statistics: http://www.alz.co.uk/research/statistics
Ammann, E. M., et al., (2013). omega-3 fatty acids and domain-specific cognitive aging: secondary analyses of data from WHISCA. Neurology, 81(17), 1484-1491.
Andrieu, S., et al., (2017). Effect of long-term omega 3 polyunsaturated fatty acid supplementation with or without multidomain intervention on cognitive function in elderly adults with memory complaints (MAPT): a randomised, placebo-controlled trial. Lancet Neurol, 16(5), 377-389.
Aridi, Y. S., Walker, J. L., & Wright, O. R. L. (2017). The Association between the Mediterranean Dietary Pattern and Cognitive Health: A Systematic Review. Nutrients, 9(7). doi: 10.3390/nu9070674
Artero, S. et al., (2008). Risk profiles for mild cognitive impairment and progression to dementia are gender specific. J Neurol Neurosurg Psychiatry, 79(9), 979-984.
Assaf, G., & Tanielian, M. (2018). Mild cognitive impairment in primary care: a clinical review. Postgrad Med J, 94(1117), 647-652.
Au, B., Dale-McGrath, S., & Tierney, M. C. (2017). Sex differences in the prevalence and incidence of mild cognitive impairment: A meta-analysis. Ageing Res Rev, 35, 176-199. doi:10.1016/j.arr.2016.09.005
Bae, J. B., et al., (2018). Impact of Mild Cognitive Impairment on Mortality and Cause of Death in the Elderly. J Alzheimers Dis, 64(2), 607-616.
Baleztena, J., et al., (2018). Association between cognitive function and supplementation with omega-3 PUFAs and other nutrients in >/=75 years old patients: A randomized multicenter study. PLoS One, 13(3), e0193568.
Barberger-Gateau, P., et al., (2007). Dietary patterns and risk of dementia: the Three-City cohort study. Neurology, 69(20), 1921-1930.
Barnard, N. D., Bunner, A. E., & Agarwal, U. (2014). Saturated and trans fats and dementia: a systematic review. Neurobiol Aging, 35 Suppl 2, S65-73.
Barnes, D. E., & Yaffe, K. (2011). The projected effect of risk factor reduction on Alzheimer's disease prevalence. Lancet Neurol, 10(9), 819-828.
Barnett, J. H., et al., (2016). The Paired Associates Learning (PAL) Test: 30 Years of CANTAB Translational Neuroscience from Laboratory to Bedside in Dementia Research. Curr Top Behav Neurosci, 28, 449-474.
Basambombo, L. L., et al., (2017). Use of Vitamin E and C Supplements for the Prevention of Cognitive Decline. Ann Pharmacother, 51(2), 118-124.
Benner, B. A., et al., (2018). Standard Reference Material (SRM) 2378 fatty acids in frozen human serum. Certification of a clinical SRM based on endogenous supplementation of polyunsaturated fatty acids. Analytical and Bioanalytical Chemistry, 410(9), 2321-2329. doi: 10.1007/s00216-017-0841-5
Bernstein, P. S., et al., (2016). Lutein, zeaxanthin, and meso-zeaxanthin: The basic and clinical science underlying carotenoid-based nutritional interventions against ocular disease. Prog Retin Eye Res, 50, 34-66. doi:10.1016/j.preteyeres.2015.10.003
Berti, V., et al., Mosconi, L. (2015). Nutrient patterns anti brain biomarkers of Alzheimer's disease in cognitively normal individuals. J Nutr Health Aging, 19(4), 413-423. doi: 10.1007/s12603-014-0534-0
Beydoun, M. A. et al., (2007). Plasma n-3 fatty acids and the risk of cognitive decline in older adults: the Atherosclerosis Risk in Communities Study. Am J Clin Nutr, 85(4), 1103-1111. doi: 10.1093/ajcn/85.4.1103
Billingsley, H. E., & Carbone, S. (2018). The antioxidant potential of the Mediterranean diet in patients at high cardiovascular risk: an in-depth review of the PREDIMED. Nutr Diabetes, 8(1), 13. doi:10.1038/s41387-018-0025-1
Boccardi, V. et al., (2016). Vitamin E family: Role in the pathogenesis and treatment of Alzheimer's disease. Alzheimers Dement (N Y), 2(3), 182-191.
Bone, R. A., & Landrum. J. T. (1984). Macular pigment in Henle fiber membranes: a model for Haidinger's brushes. Vision Res, 24(2), 103-108.
Bouayed, J., & Bohn, T. (2012). Dietary Derived Antioxidants: Implications on Health, Nutrition, Well-Being and Health. Retrieved from https://www.intechopen.com/books/nutition-well-being-and-health/dietary-derived-antioxidants-implication-on-health.
Bovier, E. R., & Hammond, B. R. (2015). A randomized placebo-controlled study on the effects of lutein and zeaxanthin on visual processing speed in young healthy subjects. Arch Biochem Biophys, 572, 54-57.
Bowman, G. L., et al., (2013). Plasma omega-3 PUFA and white matter mediated executive decline in older adults. Front Aging Neurosci, 5, 92.
Brigelius-Flohé, R. (2009). Vitamin E: the shrew waiting to be tamed. Free Radic Biol Med, 46(5), 543-554.
Britton, G. (1995). Carotenoids. Volume 1B: Spectroscopy (G. Britton Ed.). Basel: Birkhauser Verlag.
Brookmeyer, R. et al., (2007). Forecasting the global burden of Alzheimer's disease. Alzheimers Dement, 3(3), 186-191. doi:10.1016/j.jalz.2007.04.381
Brown, J. M. et al., (2017). Certification of Standard Reference Material® 968f Fat-Soluble Vitamins in Frozen Human Serum. Special Publication (NIST SP)-260-188.
Burckhardt, M. et al., (2016). Omega-3 fatty acids for the treatment of dementia. Cochrane Database Syst Rev, 4, Cd009002. doi:10.1002/14651858.CD009002.pub3
Burke, S. L., et al., (2018). Mild cognitive impairment: associations with sleep disturbance, apolipoprotein e4, and sleep medications. Sleep Med, 52, 168-176.
Cadena-Hernandez, A., et al., (2018). Longitudinal gender-specific differences in the conversion from mild cognitive impairment to Alzheimer's disease. Paper presented at the IEEE EMBS International Conference on Biomedical & Health Informatics (BHI), Las Vegas, Navada.
Calder, P. C., & Yaqoob, P. (2009). Understanding omega-3 polyunsaturated fatty acids. Postgrad Med, 121(6), 148-157. doi:10.3810/pgm.2009.11.2083
Cambridge Cognition. (2012). CANTAB Eclipse Test Administration Guide. Cambridge UK: Cambridge Cognition Limited.
Cambridge Cognition. (2016a). CANTAB Connect Research Outcome Measures v4.0. Retrieved from UK:
Cambridge Cognition. (2016b). Product Overview: CANTAB Connect Research. Retrieved from Cambridge, UK.:
Canevelli, M., et al., (2016). Spontaneous Reversion of Mild Cognitive Impairment to Normal Cognition: A Systematic Review of Literature and Meta-Analysis. J Am Med Dir Assoc, 17(10), 943-948. doi:10.1016/j.jamda.2016.06.020
Cannavale, C. N., et al., (2019). Serum Lutein is related to Relational Memory Performance. Nutrients, 11(4). doi: 10.3390/nu11040768
Chen, C. T., et al., (2009). Rapid β-oxidation of eicosapentaenoic acid in mouse brain: An in situ study. Prostaglandins, Leukotrienes and Essential Fatty Acids, 80(2), 157-163. doi:https://doi.org/10.1016/j.plefa:2009.01.005
Cheng, G., et al., (2012). Diabetes as a risk factor for dementia and mild cognitive impairment: a meta-analysis of longitudinal studies. Intern Med J, 42(5), 484-491.
Cherbuin, N., & Anstey, K. J. (2012). The Mediterranean diet is not related to cognitive change in a large prospective investigation: the PATH Through Life study. Am J Geriatr Psychiatry, 20(7), 635-639. doi:10.1097/JGP.0b013e31823032a9
Cherbuin, N., et al., (2014). Dietary Mineral Intake and Risk of Mild Cognitive Impairment The PATH through Life Project Front Aging Neurosci. 6, 4.
Collins, F. D., et al., (1971). Plasma Lipids in Human Linoleic Acid Deficiency. Nutr Metab, 13, 150-167. doi: 10.1159/000175332
Connolly, S., et al., (2014). Estimating the economic and social costs of dementia in Ireland. Dementia (London), 13(1), 5-22. doi:10.1177/1471301212442453
Copp, R. P., et al., (1999). Localization of alpha-tocopherol transfer protein in the brains of patients with ataxia with vitamin E deficiency and other oxidative stress related neurodegenerative disorders. Brain Res, 822(1-2), 80-87.
Craft, N. (2016). Tocopherols: Properties and Determination. Encyclopedia of Food and Health, 5, 309-318. doi: 10.1016/B978-0-12-384947-2.00694-2
Craft, N. E., et al., (2004). Carotenoid, tocopherol, and retinol concentrations in elderly human brain. J Nutr Health Aging, 8(3), 156-162. Retrieved from https://www.ncbi.nlm.nih.gov/pubmed/15129301
Crichton, G. E., Bryan, J., & Murphy, K. J. (2013). Dietary antioxidants, cognitive function and dementia—a systematic review. Plant Foods Hum Nutr, 68(3), 279-292.
Cunnane, S. C., et al., (2012). Plasma and brain fatty acid profiles in mild cognitive impairment and Alzheimer's disease. J Alzheimers Dis, 29(3), 691-697.
Dangour, A. D., et al., (2009). Fish consumption and cognitive function among older people in the UK baseline data from the OPAL study. J Nutr Health Aging 13(3), 198-202.
Davis, C., et al., (2015). Definition of the Mediterranean Diet; a Literature Review. Nutrients, 7(11), 9139-9153. doi:10.3390/nu7115459
de Jager, C. A., et al., (2012). Cognitive and clinical outcomes of homocysteine-lowering B-vitamin treatment in mild cognitive impairment: a randomized controlled trial. Int J Geriatr Psychiatry, 27(6), 592-600. doi: 10.1002/gps.2758
de la Rubia Orti, J. E., et al., (2018). Improvement of Main Cognitive Functions in Patients with Alzheimer's Disease after Treatment with Coconut Oil Enriched Mediterranean Diet: A Pilot Study. J Alzheimers Dis, 65(2), 577-587.
de Wilde, M. C., et al., (2017). Lower brain and blood nutrient status in Alzheimer's disease: Results from meta-analyses. Alzheimers Dement (N Y), 3(3), 416-431.
DeCarlo, C. A., et al., (2016). Vascular Health and Genetic Risk Affect Mild Cognitive Impairment Status and 4-Year Stability: Evidence From the Victoria Longitudinal Study. J Gerontol B Psychol Sci Soc Sci, 71(6), 1004-1014.
Devore, E. E., et al., (2009). Dietary intake of fish and omega-3 fatty acids in relation to long-term dementia risk. Am J Clin Nutr, 90(1), 170-176.
Devore, E. E., et al., (2010). Dietary antioxidants and long-term risk of dementia. Arch Neurol, 67(7), 819-825. doi:10.1001/archneurol.2010.144
Dong, Y., et al., (2018). Do low-serum vitamin E levels increase the risk of Alzheimer disease in older people? Evidence from a meta-analysis of case-control studies. Int J Geriatr Psychiatry, 33(2), e257-e263. doi: 10.1002/gps.4780
Douaud, G., et al., (2013). Preventing Alzheimer's disease-related gray matter atrophy by B-vitamin treatment. Proc Natl Acad Sci USA. 110(23), 9523-9528.
Droge, W. (2002). Free radicals in the physiological control of cell function. Physiol Rev, 82(1), 47-95. doi:10.1152/physrev.00018.2001
Dubois, B., & Albert, M. L. (2004). Amnestic MCI or prodromal Alzheimer's disease? Lancet Neurol., 3(4), 246-248.
Dysken, M. W., et al., (2014). Effect of vitamin E and memantine on functional decline in Alzheimer disease: the TEAM-AD VA cooperative randomized trial. JAMA, 311(1), 33-44. doi:10.1001/jama.2013.282834
Engelhart, M. J., et al., (2002). Dietary intake of antioxidants and risk of Alzheimer disease. JAMA, 287(24), 3223-3229.
Eriksdotter, M., et al., (2015). Plasma Fatty Acid Profiles in Relation to Cognition and Gender in Alzheimer's Disease Patients During Oral Omega-3 Fatty Acid Supplementation: The OmegAD Study. J Alzheimers Dis, 48(3), 805-812.
Etgen, T., et al., (2011). Mild cognitive impairment and dementia: the importance of modifiable risk factors. Dtsch Arztebl Int, 108(44), 743-750.
European Food Safety Authority. (2015). Scientific Opinion on Dietary Reference Values for vitamin E as α-tocopherol. EFSA Journal, 13(7).
European Medicines Agency (EMA) Sciences Medicines Health. (2011). Guideline on bioanalytical method validation.
Farias, S. T., et al., (2009). Progression of mild cognitive impairment to dementia in clinic-vs community-based cohorts. Arch Neurol, 66(9), 1151-1157.
Farley, A., McLafferty, E., & Hendry, C. (2011). Theories of Ageing. In The Physiological Effects ofAgeing (pp. 7-13). United Kingdom: John Wiley & Sons.
Feart, C., et al., (2016). Plasma Carotenoids Are Inversely Associated With Dementia Risk in an Elderly French Cohort. J Gerontol A Biol Sci Med Sci, 71(5), 683-688.
Feart, C., Samieri, C., & Barberger-Gateau, P. (2015). Mediterranean diet and cognitive health: an update of available knowledge. Curr Opin Clin Nutr Metab Care, 18(1), 51-62. doi:10.1097/mco.0000000000000131
Fiedor, J., & Burda, K. (2014). Potential role of carotenoids as antioxidants in human health and disease. Nutrients, 6(2), 466-488. doi:10.3390/nu6020466
Flodgren, G. M., & Berg, R. C. (2016). NIPH Systematic Reviews. In Primary and Secondary Prevention Interventions for Cognitive Decline and Dementia. Oslo, Norway: Knowledge Centre for the Health Services at The Norwegian Institute of Public Health (NIPH) Copyright (c) 2016 by The Norwegian Institute of Public Health (NIPH).
Ford, A. H., & Almeida, O. P. (2012). Effect of homocysteine lowering treatment on cognitive function: a systematic review and meta-analysis of randomized controlled trials. J Alzheimers Dis, 29(1), 133-149. doi:10.3233/JAD-2012-111739
Ford, E. S., et al., (2006). Distribution of serum concentrations of alpha-tocopherol and gamma-tocopherol in the US population. Am J Clin Nutr, 84(2), 375-383.
Freund-Levi, Y., et al., (2006). Omega-3 fatty acid treatment in 174 patients with mild to moderate Alzheimer disease: OmegAD study: a randomized double-blind trial. Arch Neurol, 63(10), 1402-1408. doi:10.1001/archneur.63.10.1402
Friedman, L., Furberg, C., & Demets, D. (2010). Fundamentals of Clinical Trials (4th ed.). New York: Springer.
Ganguli, M., et al., (2005). Alzheimer disease and mortality: a 15-year epidemiological study. Arch Neurol, 62(5), 779-784. doi:10.1001/archneur.62.5.779
Gao, Q., et al., (2018). Mild Cognitive Impairment Reversion and Progression: Rates and Predictors in Community-Living Older Persons in the Singapore Longitudinal Ageing Studies Cohort. Dement Geriatr Cogn Dis Extra, 8(2), 226-237.
Gardener, S., et al., (2012). Adherence to a Mediterranean diet and Alzheimer's disease risk in an Australian population. Transl Psychiatry, 2, e164.
Gómez-Pinilla, F. (2008). Brain foods: the effects of nutrients on brain function. Nat Rev Neurosci, 9(7), 568-578. doi:10.1038/nm2421
Goodwin, J. S., Goodwin, J. M., & Garry, P. J. (1983). Association between nutritional status and cognitive functioning in a healthy elderly population. JAMA, 249(21), 2917-2921.
Gray, S. L., et al., (2008). Antioxidant vitamin supplement use and risk of dementia or Alzheimer's disease in older adults. J Am Geriatr Soc, 56(2), 291-295.
Grimm, M. O. W., et al., (2017). Vitamin D and Its Analogues Decrease Amyloid-O (Aβ) Formation and Increase Aβ-Degradation. International Journal of Molecular Sciences, 18(12), 2764. doi:10.3390/ijmsl8122764
Grodstein, F., Chen, J., & Willett, W. C. (2003). High-dose antioxidant supplements and cognitive function in community-dwelling elderly women. Am J Clin Nutr, 77(4), 975-984. doi:10.1093/ajcn/77.4.975
Gu, Y., et al., (2010). Mediterranean diet, inflammatory and metabolic biomarkers, and risk of Alzheimer's disease. J Alzheimers Dis, 22(2), 483-492.
Gueguen, S., et al., (2002). An Isocratic Liquid Chromatographic Method with Diode-Array Detection for the Simultaneous Determination of alpha-Tocopherol, Retinol, and Five Carotenoids in Human Serum (Vol. 40).
Gupta, S., Sodhi, S., & Mahajan, V. (2009). Correlation of antioxidants with lipid peroxidation and lipid profile in patients suffering from coronary artery disease. Expert Opin Ther Targets, 13(8), 889-894. doi:10.1517/14728220903099668
Harada, C. N., et al., (2013). Normnnal cognitive aging. Clin Geriatr Med, 29(4), 737-752. doi:10.1016/j.cger.2013.07.002
Hardman, R. J., et al., (2016). Adherence to a Mediterranean-Style Diet and Effects on Cognition in Adults: A Qualitative Evaluation and Systematic Review of Longitudinal and Prospective Trials. Front Nutr, 3, 22. doi:10.3389/fnut.2016.00022
Hashimoto, M., et al., (2017). Docosahexaenoic acid: one molecule diverse functions. Crit Rev Biotechnol, 37(5), 579-597.
Hasselmo, M. E. (2011). How We Remember: Brain Mechanisms of Episodic Memory. United States of America: MIT Press.
Hayat, S. A., et al., (2014). Cognitive function in a general population of men and women: a cross sectional study in the European Investigation of Cancer-Norfolk cohort (EPIC-Norfolk). BMC Geriatr, 14, 142. doi:10.1186/1471-2318-14-1
Hayflick, L. (1998). How and why we age. Exp Gerontol, 33(7-8), 639-653.
He, C., et al., (2009). Improved spatial learning performance of fat-1 mice is associated with enhanced neurogenesis and neuritogenesis by docosahexaenoic acid. Proceedings of the National Academy of Sciences, 106(27), 11370-11375.
He, W., Goodkin, D., & Kowal, P. (2016). An Aging World: 2015. Retrieved from U.S. Government Publishing Office. Washington, D.C.:
Helmer, C., et al., (2003). Association between antioxidant nutritional indicators and the incidence of dementia: results from the PAQUID prospective cohort study. Eur J Clin Nutr, 57(12), 1555-1561. doi:10.1038/sj.ejcn.1601724
Hermann, D. M. (2016). Insufficient evidence for vitamin E in Alzheimer's disease. Alzheimers Dement (N Y), 2(3), 199-201. doi:10.1016/j.trci.2016.08.003
Hooper, C., et al., (2017). Cognitive Changes with Omega-3 Polyunsaturated Fatty Acids in Non-Demented Older Adults with Low Omega-3 Index. J Nutr Health Aging, 21(9), 988-993. doi:10.1007/s12603-017-0957-5
Hu, C., et al., (2017). The prevalence and progression of mild cognitive impairment among clinic and community populations: a systematic review and meta-analysis. Int Psychogeriatr, 29(10), 1595-1608. doi:10.1017/S1041610217000473
Huang, H. Y., & Appel, L. J. (2003). Supplementation of diets with alpha-tocopherol reduces serum concentrations of gamma- and delta-tocopherol in humans. J Nutr, 133(10), 3137-3140. doi:10.1093/jn/133.10.3137
Huang, T. L. (2010). Omega-3 fatty acids, cognitive decline, and Alzheimer's disease: a critical review and evaluation of the literature. J Alzheimers Dis, 21(3), 673-690.
Huhn, S., et al., (2015). Components of a Mediterranean diet and their impact on cognitive functions in aging. Front Aging Neurosci, 7, 132.
Institute of Medicine. (2000). Dietary reference intakes for vitamin C, vitamin E, selenium, and carotenoids. Retrieved from The National Academies Press. Washington, D.C.:
Jack, C. R., et al., (ADCS), M. o. t. A. s. D. C. S. (2008). Longitudinal MRI findings from the vitamin E and donepezil treatment study for MCI. Neurobiol Aging, 29(9), 1285-1295. doi:10.1016/j.neurobiolaging.2007.03.004
Jernerén, F., et al., (2015). Brain atrophy in cognitively impaired elderly: the importance of long-chain ω-3 fatty acids and B vitamin status in a randomized controlled trial. Am J Clin Nutr, 102(1), 215-221. doi:10.3945/ajcn.114.103283
Johnson, E. J., et al., (2008). The influence of supplemental lutein and docosahexaenoic acid on serum, lipoproteins, and macular pigmentation. American Journal of Clinical Nutrition, 87(5), 1521-1529.
Jones, J. D., Kuhn, T. P., & Szymkowicz, S. M. (2018). Reverters from PD-MCI to cognitively intact are at risk for future cognitive impairment: Analysis of the PPMI cohort. Parkinsonism Relat Disord, 47, 3-7. doi:10.1016/j.parkrldis.2017.12.006
Joshi, Y. B., & Praticò, D. (2012). Vitamin E in aging, dementia, and Alzheimer's disease. Biofactors, 38(2), 90-97. doi:10.1002/biof.195
Kang, J. H., et al., (2006). A randomized trial of vitamin E supplementation and cognitive function in women. Arch Intern Med 166(22), 2462-2468.
Kang, J. H., & Grodstein, F. (2008). Plasma carotenoids and tocopherols and cognitive function: a prospective study. Neurobiol Aging, 29(9), 1394-1403.
Kaplan, L. A., Lau, J. M., & Stein, E. A. (1990). Carotenoid composition, concentrations, and relationships in various human organs. Clin Physiol Biochem, 8(1), 1-10.
Kesse-Guyot, E., et al., (2013). Mediterranean diet and cognitive function: a French study. Am J Clin Nutr, 97(2), 369-376.
Kesse-Ouyot, E., et al., (2012). Total and specific polyphenol intakes in midlife are associated with cognitive function measured 13 years later. J Nutr, 142(1), 76-83.
Khachik, F. (2006). Distrubution and metabolism of dietary carotenoids in humans as a criterion for development of nutritional supplements. Pure and Applied Chemistry, 78(8), 1551-1557.
Khachik, F., Beecher, G. R., & Goli, M. B. (1992). Separation and identification of carotenoids and their oxidation products in the extracts of human plasma. Analytical Chemistry, 64, 2111-2122.
Kim, H. S., et al., (2014). A preliminary validity study of the cambridge neuropsychological test automated battery for the assessment of executive function in schizophrenia and bipolar disorder. Psychiatry Investig 11(4), 394-401.
Knekt, P., et al., (1994). Antioxidant vitamin intake and coronary mortality in a longitudinal population study. Am J Epidemiol, 139(12), 1180-1189.
Knight, A., Bryan, J., & Murphy, K. (2016). Is the Mediterranean diet a feasible approach to preserving cognitive function and reducing risk of dementia for older adults in Western countries? New insights and future directions. Ageing Res Rev, 25, 85-101. doi:10.1016/j.arr.2015.10.005. Epub 2015 Nov. 2.
Koepsell, T. D., & Monsell, S. E. (2012). Reversion from mild cognitive impairment to normal or near-normal cognition: risk factors and prognosis. Neurology. 79(15), 1591-1598. doi: 10.1212/WNL.0b013e31826e26b7
Kowald, A., & Kirkwood, T. B. (1996). A network theory of ageing: the interactions of defective mitochondria, aberrant proteins, free radicals and scavengers in the ageing process. Mutat Res, 316(5-6), 209-236.
Kryscio, R. J., et al., (2017). Association of Antioxidant Supplement Use and Dementia in the Prevention of Alzheimer's Disease by Vitamin E and Selenium Trial (PREADViSE). JAMA Neurol, 74(5), 567-573. doi:10.1001/jamaneurol.2016.5778
Kuipers, R. S., et al., (2010). Estimated macronutrient and fatty acid intakes from an East African Paleolithic diet. Br J Nutr, 104(11), 1666-1687.
Kweder, H., & Eidi, H. (2018). Vitamin D deficiency in elderly: Risk factors and drugs impact on vitamin D status. Avicenna J Med, 8(4), 139-146.
La Fata, G., Weber, P., & Mohajeri, M. H. (2014). Effects of vitamin E on cognitive performance during ageing and in Alzheimer's disease. Nutrients, 6(12), 5453-5472.
La Rue, A., et al., (1997). Nutritional status and cognitive functioning in a normally aging sample: a 6-y reassessment. Am J Clin Nutr, 65(1), 20-29.
Lakey-Beitia, J., et al., (2017). Anti-amyloid aggregation activity of novel carotenoids: implications for Alzheimer's drug discovery. Clin Interv Aging, 12, 815-822. doi:10.2147/cia.s134605. eCollection 2017.
Lammich, S., et al., (1999). Constitutive and regulated alpha-secretase cleavage of Alzheimers amyloid precursor protein by a disintegrin metalloprotease. Proc Natl Acad Sci USA, 96(7), 3922-3927.
Landrum, J. T., & Bone, R. A. (2001). Lutein, zeaxanthin, and the macular pigment. Arch Biochem Biophys, 385(1), 28-40. doi: 10.1006/abbi.2000.2171
Lee, L. K., et al., (2013). Docosahexaenoic acid-concentrated fish oil supplementation in subjects with mild cognitive impairment (MCI): a 12-month randomised, double-blind, placebo-controlled trial. Psychopharmacology (Berl), 225(3), 605-612.
Lefèvre-Arbogast, S., et al., (2016). Dietary B Vitamins and a 10-Year Risk of Dementia in Older Persons. Nutrients, 8(12). doi: 10.3390/nu8120761
Li, F. J., Shen, L., & Ji, H. F. (2012). Dietary intakes of vitamin E, vitamin C, and beta-carotene and risk of Alzheimer's disease: a meta-analysis. J Alzheimers Dis, 31(2), 253-258. doi:10.3233/jad-2012-120349
Li, J. Q., et al., (2016). Risk factors for predicting progression from mild cognitive impairment to Alzheimer's disease: a systematic review and meta-analysis of cohort studies. J Neurol Neurosurg Psychiaty, 87(5), 476-484.
Lieblein-Boff, J. C., et al., (2015). Exploratory Metabolomic Analyses Reveal Compounds Correlated with Lutein Concentration in Frontal Cortex, Hippocampus, and Occipital Cortex of Human Infant Brain. PLoS One, 10(8), e0136904.
Lin, K. A., et al., (2015). Marked gender differences in progression of mild cognitive impairment over 8 years. Alzheimers Dement (N Y), 1(2), 103-110.
Lloret, A., et al., (2019). The effectiveness of vitamin E treatment in Alzheimer's disease. International Journal of Molecular Sciences, 20(4), E879.
Loef, M., & Walach, H. (2012). Fruit, vegetables and prevention of cognitive decline or dementia: a systematic review of cohort studies. J Nutr Health Aging, 16(7), 626-630.
Lonn, E., et al., Investigators, H. a. H.-T. T. (2005). Effects of long-term vitamin E supplementation on cardiovascular events and cancer a randomized controlled trial. JAMA, 293(11), 1338-1347. doi:10.1001/jama.293.11.1338
Lonnerdal, B. (2010). Calcium and iron absorption—mechanisms and public health relevance. Int J Vitam Nutr Res, 80(4-5), 293-299. doi:10.1024/0300-9831/a000036
Lopez, O. L., et al., (2012). Incidence of mild cognitive impairment in the Pittsburgh Cardiovascular Health Study-Cognition Study. Neurology, 79(15), 1599-1606.
Lopez, O. L., et al., (2003). Prevalence and classification of mild cognitive impairment in the Cardiovascular Health Study Cognition Study: part 1. Arch Neurol, 60(10), 1385-1389. doi:10.1001/archneur.60.10.1385
Luchsinger, J. A., et al., (2008). Higher folate intake is related to lower risk of Alzheimer's disease in the elderly. J Nutr Health Aging, 12(9), 648-650.
Luchsinger, J. A., et al., (2003). Antioxidant vitamin intake and risk of Alzheimer disease. Arch Neurol. 60(2), 203-208.
Luck, T., et al., (2007). Mild cognitive impairment in general practice: age-specific prevalence and correlate results from the Germnnan study on ageing, cognition and dementia in primary care patients (AgeCoDe). Dement Geriatr Cogn Disor, 24(4), 307-316. doi:10.1159/000108099
Luis, C. A., Keegan, A. P., & Mullan, M. (2009). Cross validation of the Montreal Cognitive Assessment in community dwelling older adults residing in the Southeastern US. Int J Geriatr Psychiatry, 24(2), 197-201. doi: 10.1002/gps.2101
Lukaschek, K., et al., (2016). Cognitive Impairment Is Associated with a Low Omega-3 Index in the Elderly: Results from the KORA-Age Study. Dementia and Geriatric Cognitive Disorders, 42(3-4), 236-245.
Ma, F., et al., (2016). Effects of 6-Month Folic Acid Supplementation on Cognitive Function and Blood Biomarkers in Mild Cognitive Impairment: A Randomized Controlled Trial in China. J Gerontol A Biol Sci Med Sci, 71(10), 1376-1383.
Mangialasche, F., et al., (2010). High plasma levels of vitamin E forms and reduced Alzheimer's disease risk in advanced age. J Alzheimers Dis, 20(4), 1029-1037.
Mangialasche, F., et al., (2013). Serum levels of vitamin E forms and risk of cognitive impairment in a Finnish cohort of older adults. Exp Gerontol, 48(12), 1428-1435.
Mangialasche, F., et al., (2012). Tocopherols and tocotrienols plasma levels are associated with cognitive impairment. Neurobiol Aging, 33(10), 2282-2290.
Mapstone, M., et al., (2014). Plasma phospholipids identify antecedent memory impairment in older adults. Nat Med, 20(4), 415-418.
Mayne, P. E., & Burne, T. H. J. (2019). Vitamin D in Synaptic Plasticity, Cognitive Function, and Neuropsychiatric Illness. Trends Neurosci, 42(4), 293-306.
Mazereeuw, G., et al., (2012). Effects of omega-3 fatty acids on cognitive performance: a meta-analysis. NeurobiolAging, 33(7), 1482.e1417-1429.
Meagher, K. A., et al., (2013). Serum response to supplemental macular carotenoids in subjects with and without age-related macular degeneration. Br J Nutr, 110(2), 289-300. doi:10.1017/s0007114512004837. Epub 2012 Dec. 5.
Miller, E. R., et al., (2005). Meta-analysis: high-dosage vitamin E supplementation may increase all-cause mortality. Ann Intern Med, 142(1), 37-46.
Min, J. Y., & Min, K. B. (2014). Serum lycopene, lutein and zeaxanthin, and the risk of Alzheimer's disease mortality in older adults. Dement. Geriatr. Cogn Disord., 37(3-4), 246-256.
Miquel, S., et al., (2018). Poor cognitive ageing: Vulnerabilities, mechanisms and the impact of nutritional interventions. Ageing Res Rev, 42, 40-55.
Mohammadzadeh, H. N., et al., (2017). Molecular Anti-inflammatory Mechanisms of Retinoids and Carotenoids in Alzheimers Disease: a Review of Current Evidence. J Mol Neurosci, 61(3), 289-304. doi:10.1007/s12031-016-0857-x. Epub 2016 Nov. 18.
Mohanty, J. G., et al., (2008). Do red blood cell-beta-amyloid interactions alter oxygen delivery in Alzheimer's disease? Adv Exp Med Biol, 614, 29-35.
Morris, M. C., et al., (2002). Vitamin E and cognitive decline in older persons. Arch Neurol, 59(7), 1125-1132.
Morris, M. C., et al., (2005). Fish consumption and cognitive decline with age in a large community study. Arch Neurol, 62(12), 1849-1853.
Morris, M. C., et al., (2005). Relation of the tocopherol forms to incident Alzheimer disease and to cognitive change. Am J Clin Nutr, 81(2), 508-514.
Morris, M. C., et al., (2018). Nutrients and bioactives in green leafy vegetables and cognitive decline: Prospective study. Neurology, 90(3), e214-e222.
Morrison, J. H., & Baxter, M. G. (2012). The Aging Cortical Synapse: Hallmarks and Implications for Cognitive Decline. Nature reviews. Neuroscience, 13(4), 240-250.
Mozaffarian, D., & Wu, J. H. (2011). Omega-3 fatty acids and cardiovascular disease: effects on risk factors, molecular pathways, and clinical events. J Am Coll Cardiol, 58(20), 2047-2067. doi: 10.1016/j.jacc.2011.06.063
Mullan, K., et al., (2017). Serum concentrations of vitamin E and carotenoids are altered in Alzheimer's disease: A case-control study. Alzheimers Dement (N Y), 3(3), 432-439. doi:10.1016/j.trci.2017.06.006. eCollection 2017 September
Murman, D. L. (2015). The Impact of Age on Cognition. Semin Hear, 36(3), 111-121.
Nakagawa, K., et al., (2011). Amyloid beta-induced erythrocytic damage and its attenuation by carotenoids. FEBS Lett, 585(8), 1249-1254.
Nasreddine, Z. S., et al., (2005). The Montreal Cognitive Assessment, MoCA: a brief screening tool for mild cognitive impairment. J Am Geriatr Soc, 53(4), 695-699.
Ngandu, T., et al., (2015). A 2 year multidomain intervention of diet, exercise, cognitive training, and vascular risk monitoring versus control to prevent cognitive decline in at-risk elderly people (FINGER): a randomised controlled trial. Lancet, 385(9984), 2255-2263. doi:10.1016/S0140-6736(15)60461-5
Niki, E. (2014). Role of vitamin E as a lipid-soluble peroxyl radical scavenger: in vitro and in vivo evidence. Free Radic Biol Med, 66, 3-12.
Nolan, J. M., et al., (2014). Macular Pigment Visual Function, and Macular Disease among Subjects with Alzheimer's Disease: An Exploratory Study (Vol. 42).
Nolan, J. M., et al., (2012). Education is positively associated with macular pigment: the Irish Longitudinal Study on Ageing (TILDA). Invest Ophthalmol Vis Sci, 53(12), 7855-7861. doi:10.1167/iovs. 11-9367
Nolan, J. M., et al., (2015). The impact of supplemental macular carotenoids in Alzheimers disease: a randomized clinical trial. J Alzheimers Dis, 44(4), 1157-1169.
Nolan, J. M., et al., (2018). Nutritional Intervention to Prevent Alzheimer's Disease: Potential Benefits of Xanthophyll Carotenoids and Omega-3 Fatty Acids Combined. J Alzheimers Dis, 64(2), 367-378. doi:10.3233/JAD-180160
Nuttall, J. R., & Oteiza, P. I. (2014). Zinc and the aging brain. Genes Nutr, 9(1), 379.
Obulesu, M., Dowlathabad, M. R., & Brambachari, P. V. (2011). Carotenoids and Alzheimer's disease: an insight into therapeutic role of retinoids in animal models. Neurochem Int, 59(5), 535-541. doi:10.1016/j.neuint.2011.04.004. Epub 2011 Jun. 7.
Olivera-Pueyo, J., & Pelegrin-Valero, C. (2017). Dietary supplements for cognitive impairment. Actas Esp Psiquiatr, 45(Supplement), 37-47.
Otaegui-Arrazola, A., et al., (2014). Diet, cognition, and Alzheimer's disease: food for thought. Eur J Nutr, 53(1), 1-23. doi: 10.1007/s00394-013-0561-3. Epub 2013 Jul. 27.
Pannese, E. (2011). Morphological changes in nerve cells during normal aging. Brain Struct Funct, 216(2), 85-89. doi:10.1007/s00429-011-0308-y. Epub 2011 Mar. 23.
Pelletier, A., et al., (2015). Mediterranean diet and preserved brain structural connectivity in older subjects. Alzheimers Dement, 11(9), 1023-1031.
Péneau, S., et al., (2011). Fruit and vegetable intake and cognitive function in the SU.VI.MAX 2 prospective study. Am J Clin Nutr, 94(5), 1295-1303.
Perkins, A. J., et al., (1999). Association of antioxidants with memory in a multiethnic elderly sample using the Third National Health and Nutrition Examination Survey. Am J Epidemiol, 150(1), 37-44. doi:10.1093/oxfordjournals.aje.a009915
Perry, A., Rasmussen, H., & Johnson, E. J. (2009). Xanthophyll (lutein, zeaxanthin) content in fruits, vegetables and corn and egg products. Journal of Food Composition and Analysis, 22(1), 9-15.
Peters, R. (2006). Ageing and the brain. Postgrad MedJ, 82(964), 84-88.
Petersen, R. C., et al., (2018). Practice guideline update summary: Mild cognitive impairment: Report of the Guideline Development, Dissemination, and Implementation Subcommittee of the American Academy of Neurology. Neurology, 90(3), 126-135. doi:10.1212/WNL.0000000000004826
Petersen, R. C., et al., (2009). Mild cognitive impairment: ten years later. Arch Neurol, 66(12), 1447-1455. doi:10.1001/archneurol.2009.266
Petersen, R. C., et al., (1999). Mild cognitive impairment: clinical characterization and outcome. Arch Neurol. 56(3), 303-308.
Petersen, R. C., et al., (2005). Vitamin E and donepezil for the treatment of mild cognitive impairment. N Engl J Med 352(23), 2379-2388.
Phillips, M. A., et al., (2012). Lower omega-3 fatty acid intake and status are associated with poorer cognitive function in older age: A comparison of individuals with and without cognitive impairment and Alzheimer's disease. Nutr Neurosci, 15(6), 271-277. doi: 10.1179/1476830512y.0000000026
Pierce, M., Cahill, S., & O'Shea, E. (2014). Prevalence and Projections of Dementia in Ireland, 2011-2046. Retrieved from Trinity College Dublin, National University of Ireland Galway, Genio. Ireland. https://www.genio.ie/system/files/publications/Dementia_Prevalence_2011_2046.pdf:
Pilchova, I., et al., (2017). The Involvement of Mg. Oxid Med Cell Longev, 2017, 6797460. doi:10.1155/2017/6797460
Prince, M., et al., (2016). World Alzheimer Report 2016. Improving Healthcare for People Living with Dementia. Coverage, Quality and Costs Now and Into the Future. London, UK.: Alzheimer's Disease International.
Qian, J., et al., (2017). APOE-related risk of mild cognitive impairment and dementia for prevention trials: An analysis of four cohorts. PLoS Med, 14(3), e1002254.
Qin, B., et al., (2017). Intake of niacin, folate, vitamin B-6, and vitamin B-12 through young adulthood and cognitive function in midlife: the Coronary Artery Risk Development in Young Adults (CARDIA) study. Am J Clin Nutr, 106(4), 1032-1040.
Rabassa, M., et al., (2015). Low Levels of a Urinary Biomarker of Dietary Polyphenol Are Associated with Substantial Cognitive Decline over a 3-Year Period in Older Adults: The Invecchiare in Chianti Study. J Am Geriatr Soc, 63(5), 938-946.
Raederstorff, D., et al., (2015). Vitamin E function and requirements in relation to PUFA. Br J Nutr, 114(8), 1113-1122. doi:10.1017/S000711451500272X
Raji, C. A., et al., (2014). Regular Fish Consumption and Age-Related Brain Gray Matter Loss. American journal of preventive medicine, 47(4), 444-451.
Randolph, C., et al., (1998). The Repeatable Battery for the Assessment of Neuropsychological Status (RBANS): preliminary clinical validity. J Clin Exp Neuropsychol, 20(3), 310-319. doi: 10.1076/jcen.20.3.310.823
Ravaglia, G., et al., (2008). Plasma tocopherols and risk of cognitive impairment in an elderly Italian cohort. Am J Clin Nutr, 87(5), 1306-1313. doi:10.1093/ajcn/87.5.1306
Raz, N. (2009). Decline and Compensation in Aging Brain and Cognition: Promises and Constraints. Neuropsychology review, 19(4), 411-414.
Reboul, E. (2019). Vitamin E intestinal absorption: Regulation of membrane transport across the enterocyte. IUBMB Life, 71(4), 416-423. doi:10.1002/iub.1955
Reisberg, B., et al., (2008). Mild cognitive impairment (MCI): a historical perspective. Int Psychogeriatr, 20(1), 18-31. doi:10.1017/S1041610207006394
Renzi, L. M., et al., (2014). Relationships between macular pigment optical density and cognitive function in unimpaired and mildly cognitively impaired older adults. Neurobiol Aging 35(7), 1695-1699.
Renzi, L. M., Hammond, B. R., & Coleman, H. D. (2013). WO 2013/009378 A1.
Rinaldi, P., et al., (2003). Plasma antioxidants are similarly depleted in mild cognitive impairment and in Alzheimer's disease. Neurobiol. Aging, 24(7), 915-919.
Roberts, R., & Knopman, D. S. (2013). Classification and epidemiology of MCI. Clin Geriatr Med, 29(4), 753-772. doi: 10.10 16/j.cger.2013.07.003
Roberts, R. O., (2010). Vegetables, unsaturated fats, moderate alcohol intake, and mild cognitive impairment. Dement Geriatr Cogn Disord, 29(5), 413-423.
Roberts, R. O., et al., (2012). The incidence of MCI differs by subtype and is higher in men: the Mayo Clinic Study of Aging. Neurology, 78(5), 342-351.
Roberts, R. O., et al., (2014). Higher risk of progression to dementia in mild cognitive impairment cases who revert to normal. Neurology, 82(4), 317-325.
Roche, W., et al., (2018). The physics of using the Heidelberg Spectralis dual-wavelength autofluorescence method for the measurement of macular pigment volume [Abstract]. Journal of Alzheimer's Disease, 64(3), 1019-1048.
Rondanelli, M., et al., (2015). Focus on Pivotal Role of Dietary Intake (Diet and Supplement) and Blood Levels of Tocopherols and Tocotrienols in Obtaining Successful Aging. Int J Mol Sci, 16(10), 23227-23249. doi:10.3390/ijms161023227
Rutjes, A. W., et al., (2018). Vitamin and mineral supplementation for maintaining cognitive function in cognitively healthy people in mid and late life. Cochrane Database Syst Rev, 12, CD011906. doi:10.1002/14651858.CD011906.pub2
Sachdev, P. S., et al., (2012). Risk profiles for mild cognitive impairment vary by age and sex: the Sydney Memory and Ageing study. Am J Geriatr Psychiatry, 20(10), 854-865. doi: 10.1097/JGP.0b013e31825461b0
Sachdev, P. S., et al., (2013). Factors predicting reversion from mild cognitive impairment to normal cognitive functioning: a population-based study. PLoS One, 8(3), e59649. doi:10.1371/journal.pone.0059649
Sachdev, P. S., et al., (2015). The Prevalence of Mild Cognitive Impairment in Diverse Geographical and Ethnocultural Regions: The COSMIC Collaboration. PLoS One. 10(11), e0142388. doi:10.1371/journal.pone.0142388
Sakai, C., et al., (2017). Fish oil omega-3 polyunsaturated fatty acids attenuate oxidative stress-induced DNA damage in vascular endothelial cells. PLoS One, 12(11), e0187934. doi:10.1371/joumnal.pone.0187934. eCollection 2017.
Salat, D. H., Kaye, J. A., & Janowsky, J. S. (1999). Prefrontal gray and white matter volumes in healthy aging and Alzheimer disease. Arch Neurol, 56(3), 338-344.
Salem, N., Jr., Vandal, M., & Calon, F. (2015). The benefit of docosahexaenoic acid for the adult brain in aging and dementia. Prostaglandins Leukot Essent Fatty Acids, 92, 15-22. doi: 10.1016/j.plefa.2014.10.003. Epub 2014 Oct. 24.
Salvati, S., et al., (2008). Eicosapentaenoic acid stimulates the expression of myelin proteins in rat brain. J Neurosci Res, 86(4), 776-784. doi: 10.1002/jnr.21537
Sano, M., et al., (1997). A controlled trial of selegiline, alpha-tocopherol, or both as treatment for Alzheimer's disease. The Alzheimer's Disease Cooperative Study. N Engl J Med, 336(17), 1216-1222. doi:10.1056/nejm199704243361704
Scahill, R. I., et al., (2003). A longitudinal study of brain volume changes in normal aging using serial registered magnetic resonance imaging. Arch Neurol, 60(7), 989-994. doi: 10.1001/archneur.60.7.989
Scarmeas, N., Anastasiou, C. A., & Yannakoulia, M. (2018). Nutrition and prevention of cognitive impairment. Lancet Neurol, 17(11), 1006-1015.
Schaefer, E. J., et al., (2006). Plasma phosphatidylcholine docosahexaenoic acid content and risk of dementia and Alzheimer disease: the Framingham Heart Study. Arch Neurol, 63(11), 1545-1550. doi:10.1001/archneur.63.11.1545
Schmidt, R., et al., (1998). Plasma antioxidants and cognitive performance in middle-aged and older adults: results of the Austrian Stroke Prevention Study. J Am Geriatr Soc, 46(11), 1407-1410.
Schürks, M., et al., (2010). Effects of vitamin E on stroke subtypes: meta-analysis of randomised controlled trials. BMJ, 341, c5702. doi:10.1136/bmj.c5702
Sen, C. K., et al., (2007). Tocotrienols: the emerging face of natural vitamin E. Vitam Horm, 76, 203-261. doi:10.1016/S0083-6729(07)76008-9
Shao, Z., et al., (2014). What do verbal fluency tasks measure? Predictors of verbal fluency performance in older adults. Frontiers in Psychology, 5, 772-782.
Sheppard, K. W., & Cheatham, C. L. (2018). Omega-6/omega-3 fatty acid intake of children and older adults in the U.S.: dietary intake in comparison to current dietary recommendations and the Healthy Eating Index. Lipids in Health and Disease, 17, 43.
Shichiri, M. (2014). The role of lipid peroxidation in neurological disorders. Journal of Clinical Biochemistry and Nutrition, 54(3), 151-160. doi: 10.3164/jcbn.14-10
Singh, B., et al., (2014). Association of mediterranean diet with mild cognitive impairment and Alzheimer's disease: a systematic review and meta-analysis. Journal of Alzheimer's Disease, 39(2), 271-282. doi: 10.3233/jad-130830
Singh, M. (2005). Essential fatty acids, DHA and human brain. Indian J Pediatr, 72(3), 239-242.
Skaper, S. D., et al., (2018). An Inflammation-Centric View of Neurological Disease: Beyond the Neuron. Frontiers in Cellular Neuroscience, 12, 72.
Sofi, F., et al., (2014). Mediterranean diet and health status: an updated meta-analysis and a proposal for a literature-based adherence score. Public Health Nutr, 17(12), 2769-2782. doi:10.1017/s1368980013003169. Epub 2013 Nov. 29.
Solfrizzi, V., et al., (2018). Nutritional interventions and cognitive-related outcomes in patients with late-life cognitive disorders: A systematic review. Neurosci Biobehav Rev, 95, 480-498. doi: 10.1016/j.neubiorev.2018.10.022
Solfrizzi, V., et al., (2017). Relationships of Dietary Patterns, Foods, and Micro- and Macronutrients with Alzheimer's Disease and Late-Life Cognitive Disorders: A Systematic Review. J Alzheimers Dis, 59(3), 815-849. doi: 10.3233/jad-170248
Stampfer, M. J., et al., (1993). Vitamin E consumption and the risk of coronary disease in women. N Engl J Med, 328(20), 1444-1449.
Strain, H. H. (1938). Leaf Xanthophylls. Washington D. C.: Carnegie Institution of Washington.
Sugasini, D., et al., (2017). Dietary docosahexaenoic acid (DHA) as lysophosphatidylcholine, but not as free acid, enriches brain DHA and improves memory in adult mice. Scientific Reports, 7(1), 11263.
Sundelöf, J., et al., (2009). Systemic tocopherols and F2-isoprostanes and the risk of Alzheimer's disease and dementia: a prospective population-based study. J Alzheimers Dis, 18(1), 71-78. doi:10.3233/JAD-2009-1125
Swainson, R., et al., (2001). Early detection and differential diagnosis of Alzheimer's disease and depression with neuropsychological tasks. Dement Geriatr Cogn Disord, 12(4), 265-280. doi:51269
Sydenham, E., Dangour, A. D., & Lim, W. S. (2012). Omega 3 fatty acid for the prevention of cognitive decline and dementia. Cochrane Database Syst Rev(6), Cd005379. doi: 10.1002/14651858.CD005379.pub3
Talaei, M., et al., (2013). Physical activity, sex, and socioeconomic status: A population based study. ARYA Atherscler, 9(1), 51-60.
Talukdar, T., et al., (2019). Nutrient biomarkers shape individual differences in functional brain connectivity: Evidence from omega-3 PUFAs. Hum Brain Mapp, 40(6), 1887-1897. doi:10.1002/hbm.24498
Thompson, J. J., Blair, M. R., & Henrey, A. J. (2014). Over the hill at 24: persistent age-related cognitive-motor decline in reaction times in an ecologically valid video game task begins in early adulthood. PLoS One, 9(4), e94215.
Tombaugh, T. N., Kozak, J., & Rees, L. (1999). Normative data stratified by age and education for two measures of verbal fluency: FAS and animal naming. Arch Clin Neuropsychol, 14(2), 167-177.
Traber, M. G. (2013). Mechanisms for the prevention of vitamin E excess. J Lipid Res, 54(9), 2295-2306. doi:10.1194/jlr.R032946
Trichopoulou, A., et al., (1995). Diet and overall survival in elderly people. BMJ, 311(7018), 1457-1460.
Trieschmann, M., et al., (2006). Macular pigment optical density measurement in autofluorescence imaging: comparison of one- and two-wavelength methods. Graefes Arch Clin Exp Ophthalmol, 244(12), 1565-1574. doi:10.1007/s00417-006-0289-3
Tucker, K. L. (2016). Nutrient intake, nutritional status, and cognitive function with ageing. Ann N Y Acad Sci, 1367(1), 38-49.
Tucker, K. L., et al., (2005). High homocysteine and low B vitamins predict cognitive decline in aging men: the Veterans Affairs Normative Aging Study. Am J Clin Nutr, 82(3), 627-635. doi:10.1093/ajcn.82.3.627
U.S. National Institute on Ageing, & World Health Organisation. (2011). Global Health and Ageing. Retrieved from http://www.who.int/ageing/publications/global_health/en/
Usoro, O. B., & Mousa, S. A. (2010). Vitamin E forms in Alzheimer's disease: a review of controversial and clinical experiences. Crit Rev Food Sci Nutr, 50(5), 414-419. doi: 10.1080/10408390802304222
Vakhapova, V., et al., (2010). Phosphatidylserine containing omega-3 fatty acids may improve memory abilities in non-demented elderly with memory complaints: a double-blind placebo-controlled trial. Dement Geriatr Cogn Disord, 29(5), 467-474.
Valls-Pedret, C., et al., (2015). Mediterranean Diet and Age-Related Cognitive Decline: A Randomized Clinical Trial. JAMA Intern Med, 175(7), 1094-1103.
van Duijn, C. M. (2004). Prospects of genetic research of mild cognitive impairment. J Intern Med, 256(3), 235-239. doi:10.1111/j.1365-2796.2004.01366.x
van het Hof, K. H., et al., (2000). Dietary factors that affect the bioavailability of carotenoids. J Nutr, 130(3), 503-506.
Vassar, R., et al., (1999). Beta-secretase cleavage of Alzheimer's amyloid precursor protein by the transmembrane aspartic protease BACE. Science, 286(5440), 735-741.
Vauzour, D. (2012). Dietary polyphenols as modulators of brain functions: biological actions and molecular mechanisms underpinning their beneficial effects. Oxid Med Cell Longev, 2012, 914273. doi:10.1155/2012/914273
Vauzour, D., et al., (2017). Nutrition for the ageing brain: Towards evidence for an optimal diet. Ageing Res Rev, 35, 222-240.
Velho, S., et al., (2008). Dietary intake adequacy and cognitive function in free-living active elderly: a cross-sectional and short-term prospective study. Clin Nuir, 27(1), 77-86.
Vishwanathan, R., et al., (2014). Macular pigment optical density is related to cognitive function in older people. Age and Ageing, 43(2), 271-275.
Vishwanathan, R., Schalch, W., & Johnson, E. J. (2016). Macular pigment carotenoids in the retina and occipital cortex are related in humans. Nutr Neurosci, 19(3), 95-101.
Voronkov, M., Braithwaite, S. P., & Stock, J. B. (2011). Phosphoprotein phosphatase 2A: a novel druggable target for Alzheimer's disease. Future Med Chem, 3(7), 821-833.
Wagner, K. H., Kamal-Eldin, A., & Elmadfa, I. (2004). Gamma-tocopherol—an underestimated vitamin? Ann Nutr Metab, 48(3), 169-188. doi: 10.1159/000079555
Wang, W., et al., (2008). Nutritional biomarkers in Alzheimer's disease: the association between carotenoids, n-3 fatty acids, and dementia severity. J. Alzheimers. Dis, 13(1), 31-38.
Waniek, S., et al., (2017). Vitamin E (α- and γ-Tocopherol) Levels in the Community: Distribution, Clinical and Biochemical Correlates, and Association with Dietary Patterns. Nutrients, 10(1). doi: 10.3390/nu10010003
Ward, A., et al., (2012). Mild cognitive impairment: disparity of incidence and prevalence estimates. Alzheimers Dement, 8(1), 14-21.
Weiser, M., Butt, C., & Mohajeri, M. (2016). Docosabexaenoic Acid and Cognition throughout the Lifespan. Nutrients, 8(2), 99.
Williamson, K. S., et al., (2002). The nitration product 5-nitro-gamma-tocopherol is increased in the Alzheimer brain. Nitric Oxide, 6(2), 221-227.
Winblad, B., et al., (2004). Mild cognitive impairment-beyond controversies, towards a consensus: report of the International Working Group on Mild Cognitive Impairment. J Intern Med, 256(3), 240-246.
Witte, A. V., et al., (2014). Long-chain omega-3 fatty acids improve brain function and structure in older adults. Cereb Cortex, 24(11), 3059-3068. doi:10.1093/cercor/bhtl63. Epub 2013 Jun. 24.
Wu, S., et al., (2015). Omega-3 fatty acids intake and risks of dementia and Alzheimer's disease: a meta-analysis. Neurosci Biobehav Rev, 48, 1-9.
Yurko-Mauro, K., Alexander, D. D., & Van Elswyk, M. E. (2015). Docosahexaenoic acid and adult memory: a systematic review and meta-analysis. PLoS One, 10(3), e0120391. doi:10.1371/journal.pone.0120391. eCollection 2015.
Yusuf, S., et al., (2000). Vitamin E supplementation and cardiovascular events in high-risk patients. N Engl J Med, 342(3), 154-160.
Zamberletti, E., et al., Parolaro, D. (2017). Lifelong imbalanced LA/ALA intake impairs emotional and cognitive behavior via changes in brain endocannabinoid system. Journal of Lipid Research, 58(2), 301-316.
Zamroziewicz, M. K., & Barbey, A. K. (2016). Nutritional Cognitive Neuroscience: Innovations for Healthy Brain Aging. Front Neurosci, 10, 240.
Zamroziewicz, M. K., & Barbey, A. K. (2017). The Meditenanean diet and healthy brain aging: innovations from nutritional cognitive neuroscience. In T. Farooqui & A. A. Farooqui (Eds.), Role of the Mediterranean Diet in the Brain and Neurodegenerative Diseases. New York, N.Y.: Elsevier Publishing Company.
Zamroziewicz, M. K., et al., (2016). Parahippocampal Cortex Mediates the Relationship between Lutein and Crystallized Intelligence in Healthy, Older Adults. Front Aging Neurosci, 8, 297.
Zaragoza-Martí, A., et al., (2018). Evaluation of Mediterranean diet adherence scores: a systematic review. BMJ Open, 8(2), e019033.
Zhang, D. M., et al., (2017). Efficacy of Vitamin B Supplementation on Cognition in Elderly Patients With Cognitive-Related Diseases. J Geriatr Psychiatry Neurol, 30(1), 50-59.
Zhang, S., et al., (2016). Cerebral mast cells contribute to postoperative cognitive dysfunction by promoting blood brain barrier disruption. Behav Brain Res, 298(Pt B), 158-166.
Zhang, Y., et al., (2016). Intakes of fish and polyunsaturated fatty acids and mild-to-severe cognitive impairment risks: a dose-response meta-analysis of 21 cohort studies. Am J Clin Nutr 103(2), 330-340.
Zhao, Y., et al., (2014). Effect of vitamin E intake from food and supplement sources on plasma α- and γ-tocopherol concentrations in a healthy Irish adult population. Br J Nutr, 112(9), 1575-1585.
Zidichouski, J. A., et al., (2009). Clinical validation of a noninvasive, Raman spectroscopic method to assess carotenoid nutritional status in humans. J Am Coll Nutr, 28(6), 687-693.
Zwilling, C. E., et al., (2019). Nutrient biomarker patterns, cognitive function, and fMRI measures of network efficiency in the aging brain. Neuroimage, 188, 239-251.
Zygouris, S., & Tsolaki, M. (2015). Computerized cognitive testing for older adults: a review. Am J Alzheimers Dis Other Demen, 30(1), 13-28.

1. A method of improving the cognitive function of a cognitively healthy human adult, the method comprising administering to the human adult an effective amount of a composition comprising lutein, zeaxanthin and meso-zeaxanthin and at least one omega-3 fatty acid, over a period of at least 24 months, the improved cognitive function being a detectable improvement in at least one of: spatial working memory as measured by the CANTAB Connect Research software SWM task; and reduced reaction time as measured by the CANTAB Connect Research Software RTI task.

2. The method according to claim 1, wherein the said at least one omega-3 fatty acid comprises docosahexaenoic acid.

3. The method according to claim 1, wherein the said at least one omega-3 fatty acid is provided as a fish oil component in the composition.

4. The method according to claim 1, wherein the said at least one omega-3 fatty acid comprises at least two fatty acids.

5. The method according to claim 1, wherein the omega-3 fatty acid comprises docosahexaenoic acid and eicosapentaenoic acid.

6. The method according to claim 1, wherein the said at least one omega-3 fatty acid is provided as the free acid, as a salt or as a triglyceride.

7. The method according to claim 6, wherein the salt comprises a monocation.

8. The method according to claim 7, wherein the salt comprises a metal monocation.

9. The method according to claim 1, wherein the composition is formulated to be suitable for oral consumption and is administered orally.

10. The method according to claim 1, wherein the composition is in unit dose form.

11. The method according to claim 10, wherein each unit dose of composition comprises 10 mgs meso-zeaxanthin, 10 mgs lutein, 2 mgs zeaxanthin, and fish oil.

12. The method according to claim 10, wherein each unit dose of composition comprises 15 mgs meso-zeaxanthin, 5 mgs lutein, 1 mg zeaxanthin, and fish oil.

13. The method according to claim 10, wherein each unit dose of the composition comprises between 20 mgs and 2 grams of docosahexaenoic acid.

14. The method according to claim 1, wherein the composition further comprises one or more of the following: acidity regulators; anticaking agents including sodium aluminosilicate, calcium or magnesium carbonate, calcium silicate, sodium or potassium ferricyanide; antioxidants including vitamin E, vitamin C, and polyphenols; colorings including artificial colorings FD&C Blue No. 1, Blue No. 2, Green No. 3, Red No. 40, Red No. 3, Yellow No. 5 and Yellow No. 6; and natural colorings caramel, annatto, cochineal, betanin, turmeric, saffron, paprika; color retention agents; emulsifiers; flavours; flavour enhancers; preservatives; stabilizers; sweeteners and thickeners.

15. The method according to claim 1, wherein the composition is in the form of a tablet or capsule.

16. The method according to claim 1, wherein the administered composition consists essentially of lutein, zeaxanthin, meso-zeaxanthin, at least one omega-3 poly-unsaturated fatty acid or corresponding salt or triglyceride, and a diluent, carrier or excipient.

17. The method according to claim 16, wherein the omega-3 poly-unsaturated fatty acid or corresponding salt or triglyceride is DHA or a salt or triglyceride of DHA.

18. The method according to claim 1, wherein the composition comprises each of lutein, zeaxanthin, meso-zeaxanthin, and DHA and/or EPA, the composition being formulated in unit dose form for daily oral administration.

19. The method according to claim 1, wherein the improvement in cognitive function is a detectable improvement in the spatial working memory task in one or more of the following: a reduction in Total Errors at stage 6; a reduction in Between Errors at stage 8; a reduction in Total Errors at stage 8; and a reduction in Total Errors combined for stages 4-8.

20. The method according to claim 1, wherein the composition is administered at least two days a week.

21. A method of causing a detectable improvement in spatial working memory and/or reaction time in a cognitively healthy human adult, as measured by the CANTAB Connect Research software, the method comprising administering to the human adult an effective amount of a composition comprising lutein, zeaxanthin, meso-zeaxathin and at least one omega-3 fatty acid, over a period of at least 24 months.

22. The method according to claim 19, wherein performance of the method results in a reduction in the number of Total Errors at stage 8 in the SWM test in the range 20-25%.

23. The method according to claim 19, wherein performance of the method results in a reduction in the simple reaction time in the RTI test in the range 4.5-5-5%.
